(19) United States
(12) Patent Application Publication
(10) Pub. No.: US 2011/0178396 A1
(43) Pub. Date: Jul. 21, 2011

(54) Nicotinamide Derivatives

(75) Inventors: Andrew Katsifis, New South Wales (AU); Ivan Greguric, New South Wales (AU)

(73) Assignee: CRC FOR BIOMEDICAL IMAGING DEVELOPMENT LTD., Bundoora (AU)

(21) Appl. No.: 12/989,257

(22) PCT Filed: Apr. 22, 2009
(86) PCT No.: PCT/AU2009/000504

§ 371 Date: Apr. 8, 2011

(30) Foreign Application Priority Data
Apr. 22, 2008 (AU) 2008904989

(51) Int. Cl. A61B 6/00; C07D 213/79; C07D 213/80; C07D 215/36; A61K 49/00

(52) U.S. Cl. 600/426; 546/326; 546/318; 546/170; 546/319; 546/327; 424/9.1

(57) ABSTRACT

A compound comprising a pyridine carboxamide structure, for use in imaging or treating melanoma, is described. An aromatic ring in the structure is substituted with a radiohalogen atom and the substitution on the amide nitrogen atom is such that the compound binds to melanin.

### TECHNICAL FIELD

The present invention relates to synthesis and use of nicotinamide derivatives.

### BACKGROUND OF THE INVENTION

Malignant melanoma is a very aggressive cancer and despite the increasing incidence of this disease and compared to advances in other areas of cancer, there are still no effective treatments available although early detection and improved diagnostic methods have considerably decreased mortality rates over the last decade. A key feature of melanoma tumours is the extensive pigmentation present in most tumour cells thus to making it a very attractive target for both diagnosis and treatment. A suitable treatment system therefore may optimise uptake to cells containing melanin, thus providing a selective mechanism by which a significant target to non-target ratio could be achieved.

Preclinical investigations with a number of melanin targeting radiopharmaceuticals based on benzamides, demonstrated selective uptake in melanoma tumour bearing mice (Coenen, H. H. et al., J. Lab. Com. Radiopharm 1995, 37:260-62; Labarre, P. et al., Eur. J. Nucl. Med. 1999, 26:494-98; John, C. S. et al., J. Nucl. Med. 1993, 34:2169-75; Pham, T. Q. et al., J. Med. Chem. 2007, 50:3561-72). Moreover, the iodobenzamides [¹²³I]BZA (Michelot, et al., J. Nucl. Med. 1993, 34:1260-66; Brandau, W. et al., J. Nucl. Med. 1996, 37:1865-71) [¹²³I]BZA2 (Moins, N. et al., Eur. J. Nucl. Med. Mol. Imaging 2002, 29:1478-84; Sillaire-Houtmann, I. et al., J. Fr. Ophtalmol 2004, 27:34-39) [¹²³I]IBZM (Larisch, R. et al., J. Nucl. Med. 1998, 39:996-1001), the iodobenzylamine ERC9 (Salopek, T. G. et al., Eur. J. Nucl. Med. 2001, 28:408-17) have been evaluated in melanoma patients resulting in excellent detection of melanoma and its metastases with high sensitivity and selectivity. These studies confirmed the efficacy of these iodinated radiopharmaceuticals as useful imaging agents in patients with cutaneous and ocular melanoma based on the selective high affinity binding to melanin containing melanocytes. A key feature in the development of any successful radiopharmaceutical for either imaging or therapy is the high selective accumulation of the radiopharmaceutical in the target tumour with a concomittent low accumulation (fast clearance) of the radiopharmaceutical or its metabolites form all other tissue ie maximize the target to non-target ratios. The incorporation of suitable molecular fragments onto melanin seeking compounds could provide such radiopharmaceuticals.

FDG (¹⁸F-fluorodeoxyglucose) is currently the most widely used radiopharmaceutical for imaging melanoma. However, it is also non-specific and is taken up by inflammatory lesions and scars, reducing its efficacy as a tumour specific imaging agent. Iodine-123 radiolabelled SPECT benzamides, such as BZA and BZA₂, developed in the last 15 years, have not appeared in the clinical circuit due to wide availability of PET FDG and the relatively higher cost of iodine-123.

Iodinated benzamides reported in the literature are shown below.

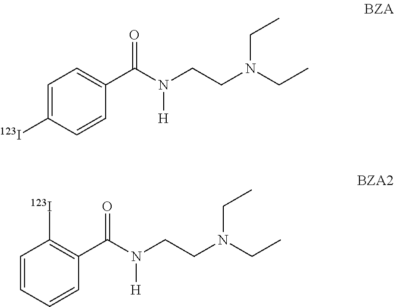

To date there is only one reported ¹⁸F labelled benzamide (structure A below) that has affinity for the melanin pigment with demonstrated efficacy in melanoma imaging as Is demonstrated in melanoma tumour bearing mice. This structure is based on the iodinated benzamide BZA and was recently reported (Garg, S. et al., J. Lab. Comd. 2007 S80. Garg, S. et al., J. Nucl. Med 2007, 5, 18P Abstract 61).

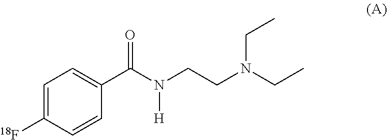

The reported synthesis and biological efficacy of this molecule includes a three step synthesis, shown below, requiring 2-3 hours and providing an overall radiochemical yield of 18%.

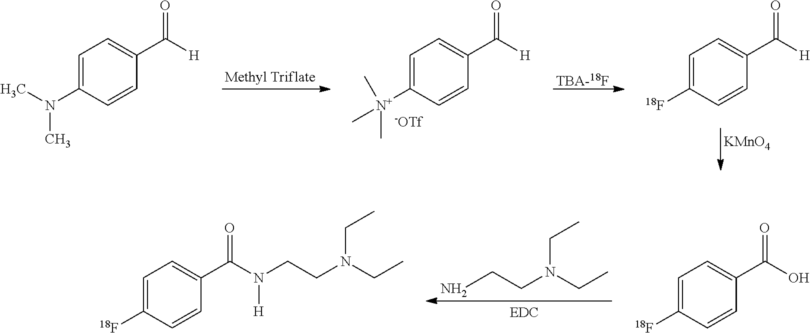

This synthesis (as reported in the literature) is characterised by an 18±5% radiochemical yield, three radiolabelling steps with purification, difficulty to automate and a synthesis time 3 h. The reported tumour uptake at 2 h is 6.5% ID/g, Typical Tumour: Blood ratio=12 (Garg, S. et al J. Lab. Comd. 2007 S80) and the Society of Nuclear Medicine (2007, Ref Garg, S. et al JNM 2007, 5, 18P Abstract 61).

A disadvantage with the known [¹⁸F] radiolabelled benzamides described above is that the radiosynthesis introduces the radiolabel before the final synthesis step. This lengthens the time during the synthesis in which a radiolabelled species is used, which is a disadvantage when the radiolabel has short half-life.

There is therefore a need for an improved reagent for use in imaging melanoma tumours, and for an improved synthesis for making them.

### Object of the Invention

It is the object of the present invention to substantially overcome or at least ameliorate one or more of the above disadvantages whilst maintaining the high biological affinity of the compound for melanin. It is another object to at least partially satisfy the above need.

### SUMMARY OF THE INVENTION

In a broad form the present invention relates to a pyridine carboxamide compound for imaging or treating melanoma, said compound being capable of binding to melanin and comprising a radiohalogen.

In a first aspect of the invention there is provided a compound for imaging or treating melanoma, said compound comprising a pyridine carboxamide structure wherein an aromatic ring in the structure is substituted with a radiohalogen atom and wherein the substitution on the amide nitrogen atom is such that the compound binds to melanin.

The following options may be used in conjunction with the first aspect, either individually or in any suitable combination.

The pyridine carboxamide structure may be a pyridine-3-carboxamide structure.

The aromatic ring that is substituted with the radiohalogen atom may be the pyridine ring of the pyridine carboxamide structure. It may be a ring fused with the pyridine ring. It may be a ring substituted onto the pyridine ring. It may be a ring in some other part of the molecule.

The substitution on the amide nitrogen atom may comprise at least one aminoalkyl group. It may comprise a hydrogen atom and a tertiary aminoalkyl group.

The substitution on the amide nitrogen atom may be such that the amide nitrogen is a member of a saturated ring structure having a second nitrogen atom in the ring. The saturated ring structure may for example be a piperazine ring structure. The second nitrogen atom may be substituted with an arylalkyl group. The arylalkyl group may be for example a phenylmethyl group. The aromatic ring that is substituted with the radiohalogen atom may be the aryl group of the arylalkyl group or it may be the pyridine ring of the pyridine carboxamide structure. In an example, the substitution on the amide nitrogen atom is such that the amide nitrogen is a member of a piperazine ring structure wherein the non-amide nitrogen of the piperazine ring structure is substituted with a phenylmethyl group having the radiohalogen atom on the 4 position of the phenyl ring.

The radiohalogen atom may be selected from the group consisting of ¹⁸F, ¹²³I, ¹²⁴I, ¹²⁵I, ¹³¹I and ⁷⁶Br. In general, if the radiohalogen is ¹³¹I the compound may be suitable for treatment of melanomas, if the radiohalogen is ¹⁸F, ⁷⁶Br or ¹²⁴I the compound may be suitable for imaging melanomas using PET and if the radiohalogen is ¹²³I or ¹³¹I the compound may be suitable for imaging melanomas using SPECT. If the radiohalogen is ¹²⁵I, the compound may be suitable for biochemical studies (e.g. in research or for use in a radioimmunoassay).

The pyridine carboxamide structure may be a pyridine-3-carboxamide structure.

The pyridine ring of the pyridine carboxamide structure may be fused with a benzene ring to form a quinoline ring system.

The compound may have structure

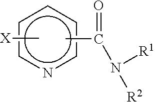

wherein X is a radiohalogen atom and R¹ and R² are independently hydrogen, an alkyl group, an aryl group or an alkylamine group, such that the compound is capable of binding to melanin.

The compound may have structure

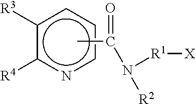

wherein:
X is a radiohalogen atom,
R¹ and R² together with the amide nitrogen form a piperazine ring, said piperazine ring being substituted with a benzyl group on the non-amide nitrogen such that the compound is capable of binding to melanin, wherein the radiohalogen atom is attached to the benzyl group; and
R³ and R⁴ together form a ring fused with the pyridine ring.

In an embodiment there is provided a compound for imaging or treating melanoma, said compound comprising a pyridine carboxamide structure, e.g. a pyridine-3-carboxamide structure, wherein the pyridine ring of the pyridine carboxamide structure is substituted with a radiohalogen atom and wherein the substitution on the amide nitrogen atom comprises comprise at least one aminoalkyl group such that the compound binds to melanin.

In another embodiment there is provided a compound for imaging or treating melanoma, said compound comprising a pyridine carboxamide structure, e.g. a pyridine-3-carboxamide structure, wherein the pyridine ring of the pyridine carboxamide structure is substituted with a radiohalogen atom selected from the group consisting of ¹⁸F, ¹²³I, ¹²⁴I, ¹²⁵I, ¹³¹I and ⁷⁶Br and wherein the substitution on the amide nitrogen atom comprises at least one aminoalkyl group such that the compound binds to melanin.

In another embodiment there is provided a compound for imaging or treating melanoma, said compound comprising a pyridine carboxamide structure, e.g. a pyridine-3-carboxamide structure, wherein the substitution on the amide nitrogen atom is such that the amide nitrogen is a member of a saturated ring structure having a second nitrogen atom in the ring, said second nitrogen atom being substituted with an arylalkyl group and the aryl group of said arylalkyl group being substituted with a radiohalogen atom.

In another embodiment there is provided a compound for imaging or treating melanoma, said compound comprising a pyridine carboxamide structure, e.g. a pyridine-3-carboxamide structure, wherein the substitution on the amide nitrogen atom is such that the amide nitrogen is a member of a saturated ring structure having a second nitrogen atom in the ring, said second nitrogen atom being substituted with an arylalkyl group and the aryl group of said arylalkyl group being substituted with a radiohalogen atom selected from the group consisting of ¹⁸F, ¹²³I, ¹²⁴I, ¹²⁵I, ¹³¹I and ⁷⁶Br.

Thus in an embodiment there is provided a compound for treating melanoma, said compound comprising a pyridine carboxamide structure, e.g. a pyridine-3-carboxamide structure, wherein an aromatic ring in the structure is substituted with an ¹³¹I atom and wherein the substitution on the amide nitrogen atom is such that the compound binds to melanin.

In another embodiment there is provided a compound for imaging melanoma using PET, said compound comprising a pyridine carboxamide structure, e.g. a pyridine-3-carboxamide structure, wherein an aromatic ring in the structure is substituted with an ¹⁸F, ⁷⁶Br or ¹²⁴I atom and wherein the substitution on the amide nitrogen atom is such that the compound binds to melanin.

In another embodiment there is provided a compound for imaging melanoma using SPECT, said compound comprising a pyridine carboxamide structure, e.g. a pyridine-3-carboxamide structure, wherein an aromatic ring in the structure is substituted with an ¹²³I or ¹³¹I atom and wherein the substitution on the amide nitrogen atom is such that the compound binds to melanin.

In another embodiment there is provided a compound for biochemical studies of melanoma, said compound comprising a pyridine carboxamide structure, e.g. a pyridine-3-carboxamide structure, wherein an aromatic ring in the structure is substituted with an ¹²⁵I atom and wherein the substitution on the amide nitrogen atom is such that the compound binds to melanin.

In a second aspect of the invention there is provided a process for making a compound for imaging or treating melanoma comprising the step of treating a precursor comprising a leaving group so as to replace said leaving group with a radiohalogen atom, said precursor comprising a pyridine carboxamide structure wherein an aromatic ring in the structure is substituted with said leaving group and wherein the substitution on the amide nitrogen atom is such that the compound binds to melanin.

The precursor may have the structure of the compound described in the first aspect (including any of the options and embodiments thereof), with exception the radiohalogen atom of the compound described in the first aspect is replaced by the leaving group.

The following options may be used in the second aspect, either individually or in any suitable combination.

The leaving group may be a non-radioactive halogen atom. The non-radioactive halogen atom may be chlorine or bromine. The leaving group may be a nitro group. It may be some other leaving group.

The step of treating the precursor may comprise treating the precursor with a complex of M⁺[¹⁸F⁻]. This may generate “naked fluoride” capable of undergoing nucleophilic substitution. The complex may comprise a phase transfer catalyst or an M⁺ ion complexing agent such as Kryptofix_{2.2.2} or a crown ether or M⁺ may be sufficiently large, such as cesium or tetrabutyl ammonium, to effectively induce nucleophilic substitution by the [¹⁸F]fluoride ion. Kryptofix_{2.2.2} is 4,7,13,16,21,24-hexaoxa-1,10-diazabicyclo[8.8.8]-hexacosane (C₁₈H₃₆N₂O₆). Thus the step of treating the precursor may comprise treating the precursor with a complex of M⁺[¹⁸F⁻], wherein M⁺ is a metal ion which is either sufficiently large to allow substitution of the leaving group with ¹⁸F⁻ or is complexed with a complexing agent so as to allow substitution of the leaving group with ¹⁸F⁻.

Alternatively an organometallic derivative, such as a trialkyl tin containing intermediate, may be used to direct a radiohalogen onto a molecule for imaging or treating melanoma. This may be particularly useful in cases where nucleophilic addition is not possible, for example to make compound 23 or when a particular nicotinamide precursor is not available for nucleophilic substitution. This route may be used for incorporating radiohalogens such radiofluorine, radiobromine or radioiodine via electrophilic substitution (X⁺ equivalent type reaction).

The step of treating the precursor may comprise the steps of:
substituting the non-radioactive halogen atom by an organometallic group, such as an alkyl tin group; and
substituting the organometallic group by the radiohalogen atom.

In this instance, the substitution of the organometallic group is by an electrophilic group, i.e. it comprises reacting the alkyl tin substituted compound with reagent which is a source of X⁺ (where X is a halogen). Thus the radiohalogen atom in the reagent should be in an electrophilic form. Suitable reagents may be produced by the action of an oxidising agent such chloramine-T (N-chlorotosylamide sodium salt), peracetic acid, hydrogen peroxide, iodogen (1,3,4,6-tetrachloro-3a,6a-diphenylglucoluril) or N-chlorosuccinimide on a M⁺X⁻ salt of the radiohalogen, wherein X is ¹²³I, ¹²⁴I, ¹²⁵I, ¹³¹I an ⁷⁶Br or electrophilic halogen equivalent. In the case of radiolabelling with [¹⁸F]fluorine the reagent may be F₂ gas or some other source of F⁺, such as acetyl hypofluorite CH₃COOF.

The final chemical step of the process may comprise introducing the radiohalogen atom into the compound in a considerable shorter time than that required for the corresponding benzamide. It will be understood that further, non-chemical steps such as purification steps may be conducted subsequent to the final chemical step.

The radiochemical yield of the final chemical step of the process may be greater than about 50%. The radiochemical yield of the total synthesis may be higher than for the corresponding benzamides.

In an embodiment there is provided a process for making a compound for imaging or treating melanoma, said process comprising:
substituting a non-radioactive chlorine or bromine atom in a precursor by an organometallic group, such as an alkyl tin group; and
substituting the organometallic group with a radiohalogen atom;
   wherein said precursor comprises a pyridine carboxamide structure, e.g. a pyridine-3-carboxamide structure, wherein the pyridine ring of the pyridine carboxamide structure is substituted with the non-radioactive halogen atom and wherein the substitution on the amide nitrogen atom comprises comprise at least one aminoalkyl group such that the compound binds to melanin. This embodiment is applicable to the radiohalogens ⁷⁶Br and ¹²³I, ¹²⁴I, ¹²⁵I, ¹³¹I or ¹⁸F as F₂ or electrophilic fluorine.

In another embodiment there is provided a process for making a compound for imaging or treating melanoma, said process comprising:
substituting a non-radioactive chlorine or bromine atom in a precursor by an organometallic group such as an alkyl tin group; and
substituting the organometallic group with a radiohalogen;
   wherein said precursor comprises a pyridine carboxamide structure, e.g. a pyridine-3-carboxamide structure, wherein the substitution on the amide nitrogen atom is such that the amide nitrogen is a member of a saturated ring structure having a second nitrogen atom in the ring, said second nitrogen atom being substituted with an arylalkyl group and the aryl group of said arylalkyl group being substituted with the non-radioactive chlorine or bromine atom.

The invention also provides a compound for imaging or treating melanoma, said compound being made by the process of the second aspect.

In a third aspect of the invention there is provided a composition for use in treating or imaging melanoma, said composition comprising a compound according to the first aspect, or made by the process of the second aspect, together with one or more pharmaceutically acceptable carriers and/or adjuvants.

In a fourth aspect of the invention there is provided a compound according to the first aspect, or made by the process of the second aspect, or of a composition according to the third aspect, when used in treating or imaging melanoma.

In a fifth aspect of the invention there is provided a method for imaging a melanoma in a patient, said method comprising:
administering to said patient a compound according to the first aspect, or made by is the process of the second aspect, or a composition according to the third aspect;
allowing sufficient time for an imageable quantity of the compound to accumulate in said melanoma; and
imaging the melanoma.

In one embodiment there is provided a method for imaging a melanoma in a patient, said method comprising:
administering to said patient a compound according to the first aspect, or made by the process of the second aspect, wherein the radiohalogen is ¹⁸F, ⁷⁶Br or ¹²⁴I;
allowing sufficient time for a PET-imageable quantity of the compound to accumulate in said melanoma; and
imaging the melanoma using PET.

In another embodiment there is provided a method for imaging a melanoma in a patient, said method comprising:
administering to said patient a compound according to the first aspect, or made by the process of the second aspect, wherein the radiohalogen is ¹²³I or ¹³¹I;
allowing sufficient time for a SPECT-imageable quantity of the compound to accumulate in said melanoma; and
imaging the melanoma using SPECT.

In a sixth aspect of the invention there is provided a method for treating a melanoma in a patient, said method comprising administering to said patient a therapeutically effective amount of a compound according to the first aspect, or made by the process of the second aspect, wherein the radiohalogen is ¹³¹I.

In the above methods, the administering may comprise injecting. It may comprise injecting a composition, e.g. a composition according to the third aspect, comprising the compound.

In a seventh aspect of the invention there is provided use of a compound according to the first aspect, or made by the process of the second aspect, for the manufacture of a medicament for the treatment or imaging of melanoma.

In an eighth aspect of the invention there is provide the use of a compound compound according to the first aspect, or made by the process of the second aspect, in therapy.

In an embodiment the therapy comprises treatment of melanoma and the radiohalogen is ¹³¹I.

In another embodiment the therapy comprises imaging of melanoma by PET and the radiohalogen is ¹⁸F, ⁷⁶Br or ¹²⁴I.

In another embodiment the therapy comprises imaging of melanoma by SPECT and the radiohalogen is ¹²³I or ¹³¹I.

### BRIEF DESCRIPTION OF THE DRAWINGS

A preferred embodiment of the present invention will now be described, by way of an example only, with reference to the accompanying drawings wherein:

FIG. 1 is a graph showing biodistribution of [¹⁸F]MEL2;

FIG. 2 is a graph showing percent % ID/g uptake and clearance profiles of [¹⁸F]MEL2; and

FIG. 3 is a graph showing log values of uptake and clearance profiles of [¹⁸F]MEL2.

### DETAILED DESCRIPTION OF THE PREFERRED EMBODIMENTS

The present invention provides a compound for imaging or treating melanoma. In a broad form the compound is a pyridine carboxamide compound being capable of binding to melanin and comprising a radiohalogen atom. In the present specification, where mention is made of a pyridine carboxamide compound, this also includes a pharmaceutically acceptable salt thereof. It may for example have one of the structures shown below, or a pharmaceutically acceptable salt thereof:

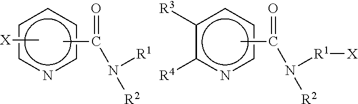

In the above structures, X is a radiohalogen atom. R¹ and R² may be such that the compound is capable of binding to melanin. They may, independently be hydrogen, an alkyl group, an aryl group, an alkylamine group or may together with the amide nitrogen form a ring structure such as a piperazine ring. The piperazine ring may be substituted on the non-amide nitrogen, e.g. with a benzyl group. R³ and R⁴ may, independently be hydrogen, an alkyl group or an aryl group, or may together form a ring fused with the pyridine ring. The fused ring may be alicyclic or may be aromatic. It may be a phenyl ring, whereby the ring structure is a quinoline ring.

In one form of the invention the compound comprises a pyridine carboxamide structure in which the pyridine ring is substituted with a radiohalogen atom and the amide nitrogen is substituted such that the compound is capable of binding to melanin. It will be understood that in this context the term “substituted with” indicates that the radiohalogen atom is directly attached to a ring atom of the pyridine ring, commonly to a ring carbon atom.

In the context of the present invention, the term “radiohalogen atom” is taken to mean a radioactive isotope of a halogen atom. It may be radioactive fluorine, bromine or iodine. It may be ¹⁸F, ¹²³I, ¹²⁵I, ¹²⁴I, ¹³¹I and ⁷⁶Br. In this specification the symbol MELx refers to radiolabelled structure x (e.g. MEL2 refers to radiolabelled 2). A prefix to this may be used to specify the nature of the radiolabel. Thus for example [¹⁸F]MEL2 will be used to refer to ¹⁸F-labelled compound 2.

The radiohalogen may be directly attached to the pyridine ring. In many embodiments it is ortho to the nitrogen of the pyridine ring, i.e. it is directly attached to C2 or C6 of the pyridine ring. Compounds according to the invention in which the radiohalogen is ¹⁸F or ¹²⁴I are commonly suitable for imaging melanoma tumours with PET, and if the radiohalogen is ¹²³I or ¹³¹I the compound may be suitable for imaging melanoma tumours with SPECT. The radiohalogen may be attached to a substituent on the amide nitrogen. It may be attached either directly to the pyridine ring or to a substituent on the amide nitrogen.

The amide group may be substituted with an aminoalkyl group. It may be a tertiary aminoalkyl group. It may be a dialkylaminoalkyl group. The two alkyl groups on the amino nitrogen (i.e. those groups that do not link the amine nitrogen to the amide nitrogen) may be the same or may be different. They may each, independently, be C1 to C6 straight chain alkyl groups or C3 to C6 branched chain or cyclic alkyl groups. They may be for example, methyl, ethyl, propyl, isopropyl, cyclopentyl, cyclohexyl or butyl. The aminoalkyl group may for example be 2-diethylaminoethyl or 4-(N-methyl-N-butylamino)-1-butyl. The aminoalkyl group may be an aminoethyl group, an aminopropyl group, an aminobutyl group or some other aminoalkyl group. The amide group may be a secondary amide group (i.e. it may have a hydrogen atom on the nitrogen atom). It may be a tertiary amide group (i.e. it may have two non-hydrogen substituents on the nitrogen atom). In the event that the amide is tertiary, it may bear an alkyl group, e.g. methyl, ethyl, propyl, isopropyl etc. It may also bear an aminoalkyl group as described above.

Alternatively the substitution on the amide nitrogen atom may be such that it forms a ring structure with the amide nitrogen. For example the carboxamide structure may be an N′-benzylpiperazinylcarbonyl substituted pyridine group.

The substitution on the amide is such that the compound binds to melanin. This enables targeting of the melanoma for therapy (imaging or treatment) applications. Thus the strength of binding to melanin should be sufficient for the required application. In addition to binding strength, clearance of the compound from non-melanin tissue is also important. The success of these compounds may reside in both the selectivity of binding to melanin tissue (tumour) and clearance from other normal tissue. Thus the binding to non-melanin tissue may be sufficiently low for the required application. The difference in binding strength to melanin tissue and to non-melanin tissue, i.e. to tumour and to normal tissue, may be sufficiently high for the required application.

The pyridine carboxamide may be a 3-pyridine carboxamide or it may be a 4-pyridine carboxamide.

In some embodiments of the invention the pyridine group is fused with a second aromatic ring. Thus the compound may comprise for example a quinoline or isoquinoline carboxamide structure or a naphthyridine carboxamide in which the pyridine ring, or one of the pyridine rings, is substituted with a radiohalogen atom and the amide nitrogen is substituted such that the compound is capable of binding to melanin. The radiohalogen atom may be on the pyridine ring of the carboxamide structure, or on an aromatic ring fused to the pyridine ring, or on an aromatic ring substituted on the pyridine ring.

The pyridine ring may have one or more other substituents. It may have hydrogen, an alkyl group or an aryl group attached to the pyridine ring on those ring carbon atoms that do not bear either a halogen atom or a carboxamide group. Each substituent may independently, for example, be hydrogen, C1 to C6 alkyl (e.g. methyl, ethyl), aryl (e.g. phenyl) or some other suitable substituent.

The invention also provides a process for making the compound of the invention. The process comprises the step of treating a precursor comprising a leaving group, such as a non-radioactive halogen so as to substitute said non-radioactive halogen with a radiohalogen. The precursor comprises a pyridine carboxamide structure, e.g. a pyridine-3-carboxamide structure, in which the pyridine ring is substituted with the leaving group and the amide nitrogen is such that the compound is capable of binding to melanin. In general, the structure of the precursor will be the same as that of the compound itself, with the exception that the halogen attached to the pyridine ring will be replaced by the leaving group. If the leaving group is a non-radioactive halogen atom, it may be the same halogen as the radiohalogen or may be a different halogen. The non-radioactive halogen may for example be non-radioactive chlorine, bromine or iodine.

The step of treating the precursor may comprise the steps of:
substituting the leaving group by an organometallic group; and
substituting the organometallic group group by the radiohalogen.

The organometallic group may be an organotin group. It may be a trialkylmetallic group, e.g. a trialkyl tin group. The alkyl group may be a C1 to C6 alkyl group, e.g. methyl, ethyl, propyl, isopropyl, butyl, cyclopentyl, or may be a mixture of alkyl groups (i.e. the three alkyl groups on the metal may not be all the same). The substitution of the leaving group may use a hexaalkyltin reagent or a trialkylstannane reagent. The trialkylstannane reagent may be for example sodium or potassium dialkylstannane. The alkyl group may be methyl, ethyl, propyl or butyl, or may be some other alkyl group. The reaction may be catalysed. It may for example be catalysed by a metal catalyst such as palladium. The metal catalyst may be ligated, for example it may be in the form of Pd(PPh₃)₄ (palladium tetrakistriphenyl phosphine) or Pd(PPh₃)₂Cl₂. The reaction may be conducted using known methods and adapted for the present starting materials.

The substitution of the alkyl tin group by the radiohalogen may use a salt of the radiohalogen ion, for example Na[¹²³I].

Alternatively step of treating the precursor may comprise treating the precursor with a complex of K[¹⁸F]. The complex may comprise a phase transfer catalyst or an M⁺ ion complexing agent such as Kryptofix_{2.2.2} or a crown ether or where M⁺ is sufficiently large such as cesium or tetrabutyl ammonium to effectively induce nucleophilic substitution by the [¹⁸F]fluoride ion. The reaction may additionally comprise heating the precursor with the complex to a temperature suitable for rapid reaction. In this context, rapid reaction may refer to reaction within 1 hour, or within 30, 20 or 10 minutes.

The final chemical step of the process may comprise introducing the radiohalogen into the compound. The final chemical step may take less than about 1 hour, or less than about 30, 20, 10 or 5 minutes. Since the radioactive decay of a radioisotope is insensitive to temperature, while reaction rates are generally accelerated by temperature, this may be achieved by heating the reaction to a suitable temperature. This enables introduction of the radiohalogen into the compounds of the invention without allowing for excessive decay of the radiohalogen.

The radiochemical yield of the final chemical step of the process, or of the process as a whole, may be greater than about 50%. It may be greater than about 60, 70 or 80%, or may be about 50 to about 95%, or about 50 to 90, 50 to 80, 50 to 70, 50 top 60, 60 to 95, 80 to 95 or 70 to 90%, e.g. about 50, 55, 60, 65, 70, 75, 80, 85, 90 or 95%, or may in some cases be greater than about 95%. It may be higher than for synthesis of the corresponding benzamides using nucleophilic substitution reaction. This may be due to the fact that the present synthesis uses a direct one step process for introducing the radiohalogen, in contrast to the three steps required for the benzamides.

The invention also provides a composition comprising a radiolabelled compound according to the invention. The composition may be suitable for injection into the patient. It may comprise one or more pharmaceutically acceptable carriers, diluents and/or adjuvants. The carriers, diluents and adjuvants must be “acceptable” in terms of being compatible with the other ingredients of the composition, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; volatile silicones; mineral oils such as liquid paraffin; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate. Typically, the carrier or carriers will form from 10% to 99.9% by weight of the compositions.

The compositions of the invention may be in a form suitable for administration by injection, in the form of a formulation suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Adjuvants typically include emollients, emulsifiers, preservatives, bactericides and buffering agents.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The invention also provides a method for imaging a melanoma in a patient. In a suitable method, a compound or composition according to the invention is administered to the patient, for example by injection. Sufficient time should then be allowed for an imageable quantity of the compound to accumulate in said melanoma. The time may depend on the patient, for example the size of the patient, and the nature of the radiolabelled compound. It may be between about 10 minutes and about 3 hours or about 10 minutes and 2 hours, 10minutes and 1 hour, 10 to 30 minutes, 30 minutes and 3 hours, 1 to 3 hours, 2 to 3 hours, 1 to 2 hours or 1.5 to 2.5 hours, e.g. about 10, 20, 30, 40 or 50 minutes, or about 1, 1.5, 2, 2.5 or 3 hours. In the context of the present specification, an imageable quantity of the compound is that quantity which, when located in the melanoma, enables imaging of the melanoma by the chosen method, for example PET or SPECT. The imageable quantity may depend on the size and nature of the melanoma, the nature of the chosen method and the nature of the radiolabelled compound. Once an imageable quantity has accumulated in the melanoma, the melanoma may then be imaged using the chose method. Use of suitable imaging methods is well known and documented.

The radiolabelled compound may be selected to be suitable for the chosen imaging method. Thus for example ¹²³I or ¹³¹I labelled compounds may be suitable for imaging by SPECT, whereas ¹⁸F, ⁷⁶Br or ¹²⁴I labelled compounds may be suitable for imaging by PET.

Particular examples of compounds according to the present invention may have the formula:

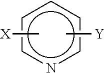

or either of the following formulae:

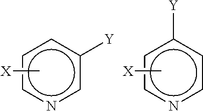

where in each case X is selected from ¹⁸F, ¹²³I, ¹²⁴I, ¹³¹I or ⁷⁶Br and Y is one of the following:

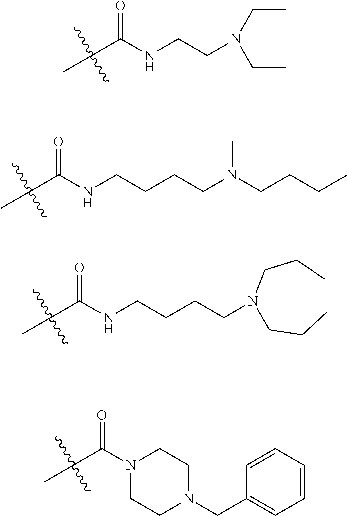

Particular examples include the following:

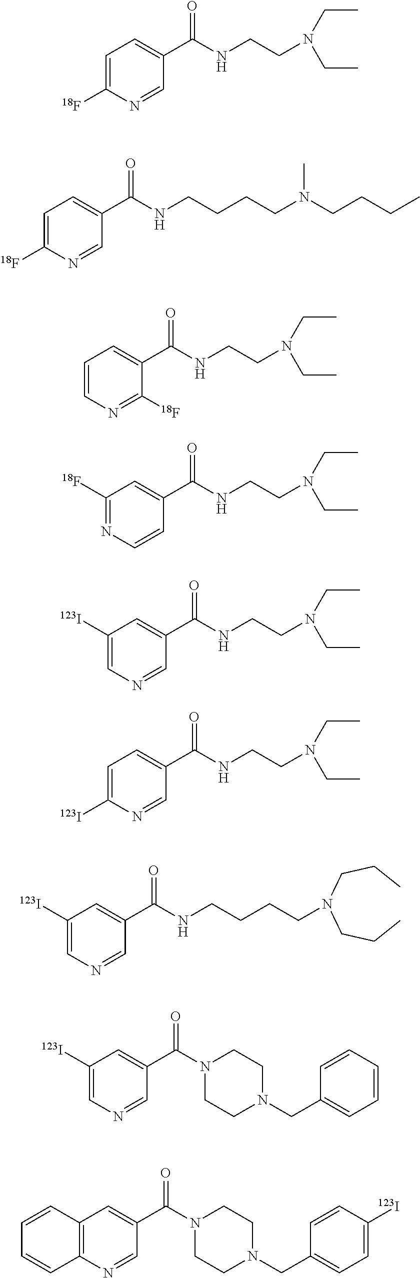

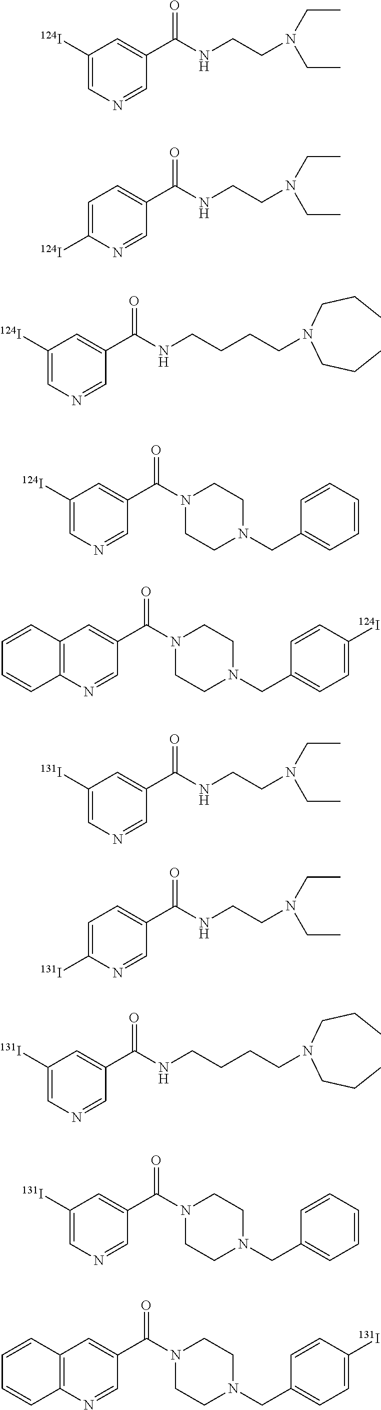

Thus the inventors have developed novel tracers for imaging melanoma based on a novel nicotinamide structure. The invention has applications in imaging melanoma tumours based on their binding to the pigment melanin. It has advantages over previously used materials due to its one step radiosynthesis method.

In this invention, a nicotinamide fragment was incorporated onto melanin binding compounds to improve the target to non-target ratios of a number of melanin seeking compounds. When subsequently radiolabelled with a radioactive isotope, such compounds can provide a radiopharmaceutical which is useful for imaging or therapy. Hence in the present invention, fluorinated (¹⁸F), brominated (⁷⁶Br) and iodinated (¹²³I, ¹²⁴I, ¹³¹I) nicotinamide analogues suitable for scintigraphic imaging with positron emission tomography (PET) or single photon emission computer tomography (SPECT) and for therapeutic purposes have been prepared.

The nicotinamide derivatives have been designed to display high tumour uptake and more rapid clearance from the body than the corresponding benzamides. A variety of alkyl- or benzylpiperazinyl side chains have been incorporated into a series of fluorinated or iodinated nicotinamides.

The significance of this invention lies with the use of nicotinamides as the basic structure of compounds which:
a) bear a radiohalogen (¹⁸F, ¹²³I, ¹²⁵I, ¹²⁴I, ¹³¹I, ⁷⁶Br) for PET and/or SPECT scintigraphic imaging or therapy; and
b) alkyl amide chains for optimum melanin binding.

An advantage of the nicotinamide over the benzamide structure is the convenience and ability to introduce a variety of radiohalogens directly onto the nicotinamide molecules in one step and in higher radiochemical yields compared to that of the benzamide derivatives.

Another advantage is the activation of the pyridine ring of the nicotinamide to nucleophilic substitution reactions. This enables a convenient and rapid method for the introduction of short lived radiohalogens such as ¹⁸F.

In comparison, the unactivated phenyl ring of benzamides requires a multistep synthesis for the incorporation of fluorine-18 onto this ring.

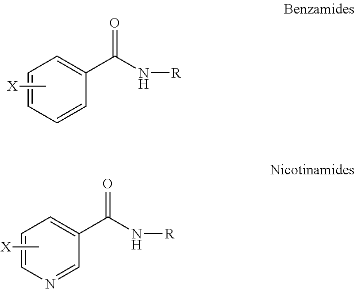

A suitable nicotinamide structure exemplified in this invention is shown below (structure B).

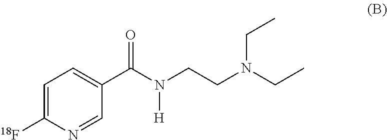

An example of the synthesis of [¹⁸F] fluoro-nicotinamides is via direct nucleophilic substitution of a halo derivative (Cl, Br, I) using typical radiofluorination reagents such as Kryptofix₂₂₂ (4,7,13,16,21,24-Hexaoxa-1-10-diazabicyclo[8.8.8]hexacosane (Kryptofix₂₂₂) in the presence of K₂CO₃ (FIG. 6) or any other amino-polyether, tetrabutyl ammonium fluoride, CsCO₃ etc. As noted above, fluorination of the molecules may comprise treating the precursor with a complex of M⁺[¹⁸F⁻] to generate “naked fluoride” capable of undergoing nucleophilic substitution. The complex may comprise a phase transfer catalyst or an M⁺ ion complexing agent such as Kryptofix_{2.2.2} or a Crown ether or M⁺ may be sufficiently large, such as cesium or tetrabutyl ammonium, to effectively induce nucleophilic substitution by the [¹⁸F]fluoride ion. This approach is illustrated below.

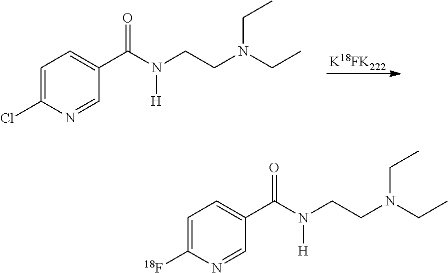

### Direct Radiofluorination of the Chloronicotinamide

The synthesis is typically characterised by a radiosynthesis time of about 40-60 minutes and a radiochemical yield greater than 50%. A typical tumour uptake at 2 h is 9% ID/g, with a tumour: blood ratio of typically about 60. The tumour to blood ratio may be at least about 20, or at least about 30, 40, 50 or 60, or about 20 to about 100, or about 20 to 80, 20 to 60, 40 to 100, 60 to 100 or 40 to 80, e.g. about 20, 30, 40, 50, 60, 70, 80, 90 or 100.

Aspects of the present invention include:
a method for imaging melanoma tumours with PET using an ¹⁸F radiolabelled nicotinamide derivative.
a method for imaging melanoma tumours with SPECT using an ¹²³I or ¹³¹I derivative or with an ¹²⁴I radiolabelled nicotinamide derivative with PET.
the invention has applications in imaging melanoma tumours based on their binding to the pigment melanin.
the ¹⁸F derivatives have advantages over previously known materials due to their one step method in radiosynthesis. This is of particular benefit in view of the short half-life of radioisotope ¹⁸F.
certain of the compounds of the invention have the element fluorine on a unique portion of the molecule which enables their simple one-step and convenient radiosynthesis whilst maintaining their uptake in melanoma tumours.
appropriately substituted alkyl chains are coupled to the nicotinamide nucleus for optimum melanin binding.
the radiolabelling processes described herein are relatively simple and rapid and can be undertaken in one step and are amenable to automation or remote radiosynthesis.

### EXAMPLES

### 1. Experimental

### Nicotinamides for [¹⁸F] Radiolabelling

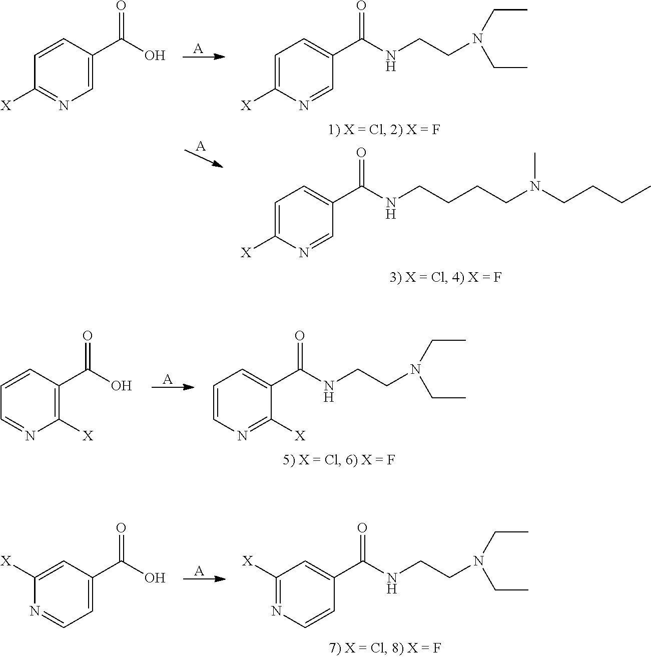

Scheme 1. Synthesis of Nicotinamides for [¹⁸F] Labelling (1-8)

A) 2-Diethylaminoethylamine or N-butyl-N-methylbutane-1,4-diamine, N-(3-dimethylaminopropyl)-N′-ethylcarbodiimide hydrochloride (EDCI), 1-hydroxybenzotriazole (HOBT), Diisopropylethyl amine (DIPEA), DMF at RT for 12 h.

Synthesis of Nicotinamides for [¹⁸F] Labelling (1-8)

### 6-Chloro-N-[2-(diethylamino)ethyl]nicotinamide (1)

6-Chloronicotinic acid (400 mg, 2.53 mmol), 2-diethylaminoethylamine (0.4 mL, 2.79 mmol), 1-hydroxybenzotriazole (HOBT, 514 mg, 3.80 mmol) and N-(3-dimethylaminopropyl)-N′-ethylcarbodiimide hydrochloride (EDCI, 730 mg, 3.80 mmol) were dissolved in DMF (Aldrich anhydrous, 5 mL). Diisopropylethyl amine (DIPEA, 0.88 mL, 5.07 mmol) was added and the resulting solution stirred overnight at room temperature. The reaction mixture was diluted with H₂O (20 mL), extracted with DCM (2×20 mL) and the combined organics washed with dilute NaHCO₃ solution (4×20 mL), dried over MgSO₄, filtered and evaporated. The compound was purified by column chromatography (EtOAc-MeOH—NH₃ 10-1-0.1) to a clear oil which crystallised on standing (513.7 mg, 79.1%). ¹H NMR (CDCl₃, 400 MHz) δ: 8.72 (d, J=2.4 Hz, 1H, ArH-2), 8.06 (dd, J=8.4, 2.4 Hz, 1H, ArH-4), 7.38 (d, J=8.4 Hz, 1H, ArH-5), 7.11 (bs, 1H, NH), 2.45 (app q, J=5.2 Hz, 2H, CONH—CH₂), 2.63 (t, J=6.0 Hz, 2H, CONH—CH₂—CH₂), 2.54 (q, J=7.2 Hz, 4H, N—(CH₂—CH₃)₂), 1.00 (t, J=7.2 Hz, 6H, N—(CH₂—CH₃)₂). ¹³C NMR (CDCl₃, 100 MHz) [symbol] :164.1 (CO), 153.9 (ArCCl), 147.9 (ArCH), 137.7 (ArCH), 129.2 (ArCCO), 124.2 (ArCH), 50.9, 46.6, 37.2 (CH₂), 11.9 (CH₃). MS (mass spectrometry): ES(+ve) m/z 258 (33%), 256 (100%, M+H).

### 6-Fluoro-N-[2-(diethylamino)ethyl]nicotinamide (2)

6-Fluoronicotinic acid (150 mg, 1.06 mmol), 2-diethylaminoethylamine (0.17 mL, 1.16 mmol), HOBT (215 mg, 1.59 mmol) and EDCI (305 mg, 1.59 mmol) were dissolved in DMF (Aldrich anhydrous, 2 mL). DIPEA, 0.37 mL, 2.12 mmol) was added and the resulting solution stirred overnight at room temperature. The reaction mixture was diluted with H₂O (10 mL), extracted with DCM (2×10 mL) and the combined organics washed with dilute NaHCO₃ solution (4×10 mL), dried over MgSO₄, filtered and evaporated. The compound was purified by column chromatography (EtOAc-MeOH—NH₃ 10-1-0.1) to a clear oil (167.1 mg, 65%). ¹H NMR (CDCl₃, 400 MHz) δ: 8.61 (d, J=2.4 Hz, 1H, ArH-2), 8.25 (dt, J=8.4, 2.8 Hz, 1H, ArH-4), 8.01 (bs, 1H, NH), 7.00 (dd, J=8.4, 2.8 Hz, 1H, ArH-5), 3.49 (app q, J=5.2 Hz, 2H, CONH—CH₂), 2.66 (t, J=6.0 Hz, 2H, CONH—CH₂—CH₂), 2.57 (q, J=7.2 Hz, 4H, N—(CH₂—CH₃)₂), 1.04 (t, J=7.2 Hz, 6H, N—(CH₂—CH₃)₂). ¹³C NMR (CDCl₃, 100 MHz) δ: 164.6 (ArCF, J=242.3 Hz) 164.1 (CO), 146.7 (ArCH, J=15.8 Hz), 140.6 (ArCH, J=8.8 Hz), 128.4 (ArCCO, J=4.5 Hz), 109.3 (ArCH, J=37.2 Hz), 51.2 46.5, 37.1(CH₂), 11.4 (CH₃). MS (mass spectrometry): ES(+ve) m/z 241 (8%), 240 (56%, M+H⁺), 168 (9%) 167 (100%).

### 6-Chloro-N-[4-(butyl(methyl)amino)butyl]nicotinamide (3)

6-Chloronicotinic acid (1.0 g, 6.34 mmol), N-butyl-N-methylbutane-1,4-diamine (1.11 g, 6.98 mmol), HOBT (1.28 g, 9.52 mmol) and EDCI (1.83 g, 9.52 mmol) were dissolved in DMF (Aldrich anhydrous, 10 mL). DIPEA (2.21 mL, 12.7 mmol) was added and the resulting solution stirred overnight at room temperature. The reaction mixture was diluted with H₂O (20 mL), extracted with DCM (2×20 mL) and the combined organics washed with dilute NaHCO₃ solution (4×20 mL), dried over MgSO₄, filtered and evaporated. The compound was purified by column chromatography (EtOAc-MeOH—NH₃ 10-1-0.1) to a clear oil which crystallised on standing (1.44 g, 76%). ¹H NMR (CDCl₃, 400 MHz) δ: 8.69 (d, J=2.4 Hz, 1H, ArH-2), 8.42 (bs, 1H, NH), 8.06 (dt, J=8.4, 2.8 Hz, 1H, ArH-4), 7.37 (d, J=8.4 Hz, 1H, ArH-5), 3.42 (q, J=5.2 Hz, 2H, CONH—CH₂), 2.36-2.28 (m, 4H, 2×CH₂), 2.15 (s, 3H, N—CH₃), 1.71-1.67 (m, 2H, CH₂), 1.68-1.60 (m, 2H, CH₂), 1.36-1.31 (m, 2H, CH₂), 1.26-1.20 (m, 2H, CH₂), 0.85 (t, J=7.25 Hz, 3H, CH₂—CH₃).

¹³C NMR (CDCl₃, 100 MHz) [symbol] :164.6 (CO), 153.6 (ArCCl), 148.0 (Ar—C—H2), 138.0 (Ar—C—H4), 129.9 (ArCCO), 124.1 (Ar—C—H5), 57.2, 57.0 (CH₂), 42.4 (N—CH₃), 40.1, 28.8, 27.4, 25.3, 20.7 (CH₂), 13.9 (CH₂—CH₃). LRMS ES(+ve) m/z 300 (32%), 298.4 (100%, M+H⁺).

### 6-Fluoro-N-[4-(butyl(methyl)amino)butyl]nicotinamide (4)

6-Fluoronicotinic acid (200 g, 1.41 mmol), N-butyl-N-methylbutane-1,4-diamine (246 mg, 1.58 mmol), HOBT (287 mg, 2.12 mmol) and EDCI (407 mg, 2.12 mmol) were dissolved in DMF (Aldrich anhydrous, 2 mL). DIPEA (0.49 mL, 2.83 mmol) was added and the resulting solution stirred overnight at room temperature. The reaction mixture was diluted with H₂O (20 mL), extracted with DCM (2×20 mL) and the combined organics washed with dilute NaHCO₃ solution (4×20 mL), dried over MgSO₄, filtered and evaporated. The compound was purified by column chromatography (EtOAc-MeOH—NH₃ 10-1-0.1) to a clear oil which crystallised on standing (216 mg, 54%). ¹H NMR (CDCl₃, 400 MHz) δ: 8.57 (d, J=2.4 Hz, 1H, ArH-2), 8.40 (bs, 1H, NH), 8.24 (dt, J=7.2, 2.4 Hz, 1H, ArH-4), 6.98 (dd, J=8.4, 2.8 Hz, 1H, ArH-5), 3.44 (q, J=5.2 Hz, 2H, CONH—CH₂), 2.41-2.32 (m, 4H, 2×CH₂), 2.19 (s, 3H, N—CH₃), 1.73-1.70 (m, 2H, CH₂), 1.69-1.63 (m, 2H, CH₂), 1.40-1.30 (m, 2H, CH₂), 1.28-1.22 (m, 2H, CH₂), 0.82 (t, J=7.2 Hz, 3H, CH₂—CH₃). ¹³C NMR (CDCl₃, 100 MHz) [symbol] :164.6 (ArCF, J=242 Hz), 164.5 (CO), 146.6 (Ar—C—H2, J=4.9 Hz), 140.7 (Ar—C—H4, J=8.7 Hz), 129.2 (ArCCO, J=4.5 Hz), 109.3 (Ar—C—H5, J=37.1 Hz), 57.2, 57.0 (CH₂), 42.3 (N—CH₃), 40.1, 28.8, 27.3, 25.2, 20.6 (CH₂), 13.9 (CH₂—CH₃). LRMS ES(+ve) m/z 283 (18%), 282 (100%, M+H⁺).

### 2-Chloro-N-[2-(diethylamino)ethyl]nicotinamide (5)

2-Chloronicotinic acid (200 mg, 1.27 mmol), N,N-diethylethylenediamine (0.2 mL, 1.39 mmol), HOBT (257 mg, 1.90 mmol) and EDCI (365 mg, 1.90 mmol) were dissolved in dry DMF (Aldrich anhydrous, 2 mL) under N₂. Diisopropylethylamine (0.44 mL, 2.54 mmol) was added to the mixture and the reaction stirred o/n at RT. Following complete consumption of the acid by TLC the reaction was diluted with H₂O (20 mL) and extracted with DCM (3×20 mL). The organics were combined, washed with water (4×20 mL), dried over MgSO4, filtered and evaporated to a crude oil, from which the title compound was purified by silica gel column chromatography using EtOAc-MeOH—NH₃ (5-1-trace) as the mobile phase as a clear oil (225.7 mg, 69.5%). ¹H NMR (CDCl₃, 400 MHz) δ: 8.43 (dd, J=4.4, 2.0 Hz, 1H, Ar—H-6), 8.10 (dd, J=7.6, 2.0 Hz, 1H, Ar—H-4), 7.32 (dd, J=7.6, 4.8 Hz, 1H, Ar—H-5), 7.26 (bs, 1H, NH), 3.05 (q, J=4.8 Hz, 2H, CONH—CH₂—CH₂), 2.64 (t, J=6.4 Hz, 2H, CONH—CH₂—CH₂), 2.55 (q, J=7.2 Hz, 4H, N—(CH₂—CH₃)₂), 1.00 (t, J=7.2 Hz, 6H, N—(CH₂—CH₃)₂). ¹³C NMR (CDCl₃, 100 MHz) δ: 164.3 (CONH), 150.7 (Ar—CH-6), 147.2 (Ar—CH—Cl), 139.7 (Ar—CH-4), 131.3 (Ar—CH—CONH), 122.6 (Ar—CH-5), 50.8 (CONH—CH₂—CH₂), 46.4 (N—(CH₂—CH₃)₂), 37.7 (CONH—CH₂—CH₂), 11.7 (N—(CH₂—CH₃)₂). LRMS ES(+) 258.3 (13%, M+H⁺), 256.2 (39%), 185.1 (32%), 183.0 (100%).

### 2-Fluoro-N-[2-(diethylamino)ethyl]nicotinantide (6)

2-Fluoronicotinic acid (200 mg, 1.41 mmol), N,N-diethylethylenediamine (0.22 mL, 1.56 mmol), HOBT (287 mg, 2.12 mmol) and EDCI (407 mg, 2.12 mmol) were dissolved in dry DMF (Aldrich anhydrous, 2 mL) under N₂. Diisopropylethylamine (0.49 mL, 2.83 mmol) was added to the mixture and the reaction stirred o/n at RT. Following complete consumption of the acid by TLC the reaction was diluted with H₂O (20 mL) and extracted with DCM (3×20 mL). The organics were combined, washed with water (4×20 mL), dried over MgSO4, filtered and evaporated to a crude oil, from which the title compound was purified by silica gel column chromatography using EtOAc-MeOH—NH₃ (5-1-trace) as the mobile phase as a clear oil (232 mg, 68%). ¹H NMR (CDCl₃, 400 MHz) δ: 8.56 (ddd, J=10.0, 7.6, 2.0 Hz, 1H, Ar—H-6), 8.29 (dt, J=4.8, 1.2 Hz, 1H, Ar—H-4), 7.63 (bs, 1H, NH), 7.33 (ddd, J=7.6, 4.8, 2.4 Hz, 1H, Ar—H-5), 3.51 (q, J=5.2 Hz, 2H, CONH—CH₂—CH₂), 2.64 (t, J=6.0 Hz, 2H, CONH—CH₂—CH₂), 2.55 (q, J=7.2 Hz, 4H, N—(CH₂—CH₃)₂), 1.03 (t, J=7.2 Hz, 6H, N—(CH₂—CH₃)₂). ¹³C NMR (CDCl₃, 100 MHz) δ: 161.4 (CONH, J=7.2 Hz), 160.1 (Ar—CH—F, J=236 Hz), 149.9 (Ar—CH-6, J=16 Hz), 143.2 (Ar—CH-4, J=3 Hz), 122.2 (Ar—CH-5, J=4.3 Hz), 116.3 (Ar—CH—CONH, J=27.5 Hz), 50.8 (CONH—CH₂—CH₂), 46.7 (N—(CH₂—CH₃)₂), 37.6 (CONH—CH₂—CH₂), 11.9 (N—(CH₂—CH₃)₂). LRMS ES(+) 241.2 (7.4%), 240.0 (53%), 168.0 (9.3%), 167.1 (100%).

### 6-Chloro-N-[2-(diethylamino)ethyl]isonicotinamide (7)

2-Chloroisonicotinic acid (200 mg, 1.26 mmol), N,N-diethylethylenediamine (0.2 mL, 1.39 mmol), HOBT (257 mg, 1.90 mmol) and EDCI (365 mg, 1.90 mmol) were dissolved in dry DMF (Aldrich anhydrous, 2 mL) under N₂. Diisopropylethylamine (0.49 mL, 2.83 mmol) was added to the mixture and the reaction stirred o/n at RT. Following complete consumption of the acid by TLC the reaction was diluted with H₂O (20 mL) and extracted with DCM (3×20 mL). The organics were combined, washed with water (4×20 mL), dried over MgSO₄, filtered and evaporated to a crude oil, from which the title compound was purified by silica gel column chromatography using EtOAc-MeOH—NH₃ (5-1-trace) as the mobile phase as a clear oil (210 mg, 65%). ¹H NMR (CDCl₃, 400 MHz) δ: 8.48 (d, J=5.2 Hz, 1H, Ar—H-6), 7.64 (s, 1H, Ar—H-3), 7.49 (dd, J=5.2, 1.6 Hz, 1H, Ar—H-5), 7.07 (bs, 1H, NH), 3.40 (q, J=5.6 Hz, 2H, CONH—CH₂—CH₂), 2.64 (t, J=5.6 Hz, 2H, CONH—CH₂—CH₂), 2.58 (q, J=6.8 Hz, 4H, N—(CH₂—CH₃)₂), 1.03 (t, J=6.8 Hz, 6H, N—(CH₂—CH₃)₂). ¹³C NMR (CDCl₃, 100 MHz) δ: 163.8 (CONH), 152.4 (Ar—CH—Cl), 150.3 (Ar—CH-6), 144.8 (Ar—CH—CONH), 122.1 (Ar—CH-3), 119.5 (Ar—CH-5), 50.5 (CONH—CH₂—CH₂), 46.7 (N—(CH₂—CH₃)₂), 37.2 (CONH—CH₂—CH₂), 11.7 (N—(CH₂—CH₃)₂).

LRMS ES(+) 258.1 (21%), 256 (64%, M+H⁺), 185 (32%), 183 (100%).

### 6-Fluoro-N-[2-(diethylamino)ethyl]isonicotinamide (8)

2-Fluoroisonicotinic acid (200 mg, 1.41 mmol), N,N-diethylethylenediamine (0.22 mL, 1.56 mmol), HOBT (287 mg, 2.12 mmol) and EDCI (407 mg, 2.12 mmol) were dissolved in dry DMF (Aldrich anhydrous, 2 mL) under N₂. Diisopropylethylamine (0.49 mL, 2.83 mmol) was added to the mixture and the reaction stirred o/n at RT. Following complete consumption of the acid by TLC the reaction was diluted with H₂O (20 mL) and extracted with DCM (3×20 mL). The organics were combined, washed with water (4×20 mL), dried over MgSO4, filtered and evaporated to a crude oil, from which the title compound was purified by silica gel column chromatography using EtOAc-MeOH—NH₃ (5-1-trace) as the mobile phase as a clear oil (261 mg, 77%). ¹H NMR (CDCl₃, 400 MHz) δ: 8.33 (d, J=5.2 Hz, 1H, Ar—H-6), 7.47 (dt, J=5.2, 1.6 Hz, 1H, Ar—H-5), 7.27 (bs, 1H, Ar—H-3), 7.08 (bs, 1H, NH), 3.48 (q, J=5.2 Hz, 2H, CONH—CH₂—CH₂), 2.65 (t, J=6.0 Hz, 2H, CONH—CH₂—CH₂), 2.57 (q, J=7.2 Hz, 4H, N—(CH₂—CH₃)₂), 1.04 (t, J=7.2 Hz, 6H, N—(CH₂—CH₃)₂). ¹³C NMR (CDCl₃, 100 MHz) δ: 164.1 (Ar—CH—F, J=239 Hz), 163.8 (CONH, J=3.5 Hz), 148.4 (Ar—CH-6, J=14.2 Hz), 147.4 (Ar—CH—CONH, J=7.2 Hz), 118.6 (Ar—CH-5, J=4.3 Hz)), 107.6 (Ar—CH-3, J=38.6 Hz), 50.9 (CONH—CH₂—CH₂), 46.6 (N—(CH₂—CH₃)₂), 37.3 (CONH—CH₂—CH₂), 11.8 (N—(CH₂—CH₃)₂). LRMS ES(+) 241.3 (14%), 240.2 (100%).

Radiopharmaceutical Preparation with K[¹⁸F]—K_{2.2.2} Complex

An aqueous [¹⁸F]fluoride solution (6-7 GBq) was added to a 10 mL vial containing anhydrous acetonitrile (1 mL), K_{2.2.2} (1 equiv) and K₂CO₃ (1 equiv). The solvent was evaporated under a stream of nitrogen at 100° C. under vacuum to produce K[¹⁸F]—K_{2.2.2} complex. This azeotropic drying was repeated twice by further addition of anhydrous acetonitrile (2×1 mL). The chloro-precursor (MEL1, 3, 5 and 7) (6 mg) was dissolved in anhydrous DMF (1 mL) and added to the dried K[¹⁸F]—K_{2.2.2} complex. The reaction was stirred and heated at 150° C. for 10 min before the reaction mixture (250 μL) was diluted with mobile phase (500 μL) and purified by semi-preparative reverse phase chromatography [Table 1]. The collected radioactive peak was evaporated in vacuo and formulated to a concentration of 1 MBq/100 μL of saline containing less than 1% ethanol for biological studies.

TABLE 1
Radiolabelling data for the [¹⁸F]MEL radiotracers

| | Purification | | | Retention | RCY |
|---|---|---|---|---|---|
| [¹⁸F]Cmpd | Solvent ^{a} | Flow rate ^{b} | | time (min) | % ^{c} |
| [¹⁸F]2 | 20/80 | 2.5 | mL/min | 19 | 35-45 |
| [¹⁸F]6 | 20/80 | 3 | mL/min | 16 | 40-55 |
| [¹⁸F]8 | 20/80 | 3 | mL/min | 16 | 30-40 |
| [¹⁸F]4 | 30/70 | 3 | mL/min | 15 | 45-55 |

^{a} Acetonitrile/Ammonium Bicarbonate solution 20 mM pH 8.
^{b} Phenomenex Bondclone C18 (10 μm, 7.8 × 300 mm).
^{c} Isolated yield (not decay corrected), specific activity 111-148 GBq/μmol.

### Nicotinamides for [^{123,124,125,131}I] Radiolabelling

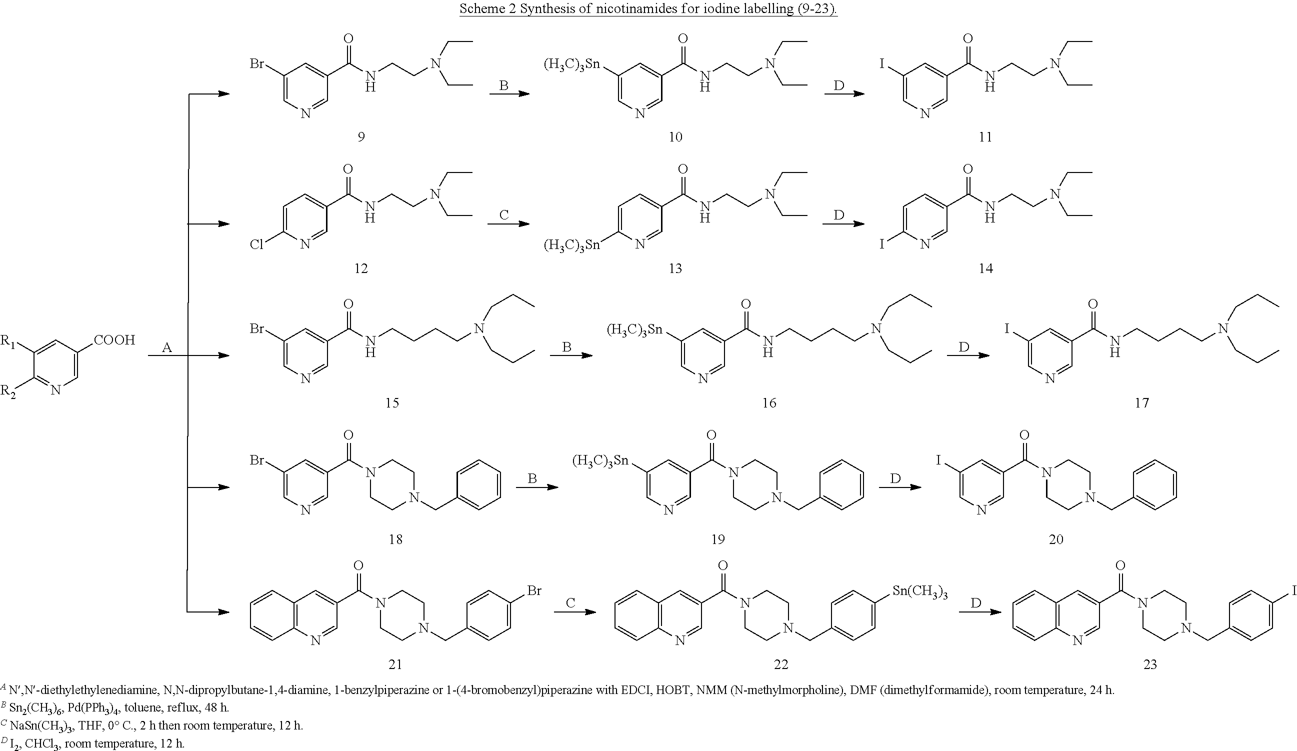

### General Procedure A for Preparation of Bromo and Chloro Nicotinamide Derivatives

To a solution of the nicotinic acid or quinoline-3-carboxylic acid (20 mmol) in dimethylformamide (DMF) (240 ml) was added the appropriate amine (20 mmol), 1-(3-dimethylaminopropyl)-3-ethylcarbodiimide hydrochloride (EDCI) (22 mmol), 1-hydroxybenzotriazole hydrate (HOBT) (22 mmol) and N-methylmorpholine (NMM) (80 mmol). The reaction was allowed to stir at room temperature for 20 h before it was diluted with water (240 ml) and then extracted into ethyl acetate (3×240 ml). The combined organic extracts were then washed with brine, dried (Na₂SO₄), and concentrated in vacuo to give the crude product. Purification by column chromatography yielded the corresponding nicotinamide derivatives.

### 5-Bromo-N-(2-(diethylamino)ethyl)nicotinamide (9)

General procedure A was applied to 5-bromonicotinic acid (4.0 g, 19.8 mmol), followed by column chromatography with CH₂Cl₂/CH₃OH (8:2) to give the title compound as an amber oil (3.9 g, 66%). ¹H NMR (CDCl₃) [symbol] :1.15, (t, J=7.2 Hz, 6H, 2×CH₃), 2.74, (q, J=7.2 Hz, 4H, 2×CH₂), 2.82, (t, J=5.6 Hz, 2H, CH₂), 3.60 (t, J=5.8 Hz, 2H, CH₂), 7.69 (br s, 1H, NH), 8.37 (t, J=2.1 Hz, 1H, Ar), 8.79 (d, J=2.2 Hz, 1H, Ar), 8.95 (d, J=1.8 Hz, 1H, Ar). ¹³C NMR (CDCl₃) [symbol] :11.7, 37.2, 51.2, 46.7, 121.0, 131.7, 137.9, 145.8, 153.1, 163.9. LRMS: ES(+ve) m/z 300(M+1). HRMS: CI(+ve) calculated for C₁₂H₁₈N₃OBr (M+H) 300.0703, found 300.0706.

### 6-Chloro-N-(2-(diethylamino)ethyl)nicotinamide (12)

General procedure A was applied to 6-chloronicotinic acid (3.0 g, 19.2 mmol), followed by column chromatography with ethyl acetate/CH₃OH (9:1) to give a yellow wax like solid (3.15 g, 64%). ¹H NMR (CDCl₃) δ: 1.04 (t, J=7.2 Hz, 6H, 2×CH₃), 2.56 (q, J=7.2 Hz, 4H, 2×CH₂), 2.66 (t, J=6.0 Hz, 2H, CH₂), 3.48 (dt, J=6.0, 6.8 Hz, 2H, CH₂), 7.07 (br s, 1H, NH), 7.41 (d, J=8.0 Hz, 1H, Ar), 8.09 (dd, J=2.4, 8.0 Hz, 1H, Ar), 8.74 (d, J=2.4 Hz, 1H Ar). ¹³C NMR (CDCl₃) [symbol] :13.2, 38.4, 47.9, 52.2, 125.5, 130.6, 139.1, 149.2, 155.2, 165.4. LRMS: ES(+ve) m/z 256 (M+1), 278 (M+Na). HRMS: CI(+ve) calculated for C₁₂H₁₉ClN₃O (M+H) 256.1211, found 256.1214.

### 5-Bromo-N-(4-(dipropylamino)butyl)nicotinamide (15)

General procedure A was applied to 5-bromonicotinic acid (4.0 g, 19.8 mmol) followed by column chromatography using CH₂Cl₂/CH₃OH (8:2) to give a wax like solid (6.0 g, 85%). ¹H NMR (CDCl₃): [symbol] :0.77 (t, J=7.2 Hz, 6H, 2×CH₃), 1.45 (m, 4H, 2×CH₂), 1.54 (m, 2H, CH₂), 1.58 (m, 2H, 2×CH₂), 2.31 (m, 4H, 2×CH₂), 2.38 (t, J=6.4 Hz, 2H, CH₂), 3.36 (dt, J=5.4, 6.4 Hz, 2H, CH₂), 8.17 (t, J=2.0 Hz, 1H, Ar), 8.21 (t, J=5.4 Hz, 1H, NH), 8.66 (d, J=2.0 Hz, 1H, Ar), 8.83 (d, J=2.0 Hz, 1H, Ar). ¹³C NMR (CDCl₃) [symbol] :12.3, 19.9, 25.5, 27.9, 40.7, 53.9, 56.4, 138.0, 146.8, 153.2, 121.0, 132.5, 164.8. LRMS: ES(+ve) m/z 357 (M+1). Anal. calculated for C₁₆H₂₆BrN₃O.0.75H₂O, C, H, N: 51.95, 7.51, 11.36, found 51.84, 6.96, 11.13.

### (4-Benzylpiperazin-1-yl)(5-bromopyridin-3-yl)methanone (18)

General procedure A was applied to 5-bromonicotinic acid (4.0 g, 19.8 mmol), followed by column chromatography using ethyl acetate/CH₃OH (9:1) to give the title compound as pale white crystals (5.58 g, 78%), mp 176-178° C. ¹H NMR (CDCl₃) [symbol] :2.44 (br s, 2H, CH₂), 2.57 (br s, 2H, CH₂), 3.48 (br s, 2H, CH₂), 3.56 (s, 2H, CH₂), 3.83 (br s, CH₂), 7.24-7.35 (m, 5H, Ar), 7.95 (t, J=2.0 Hz, 1H, Ar), 8.69 (d, J=2.0, 1H, Ar), 8.88 (d, J=2.0 Hz, 1H, Ar). ¹³C NMR (CDCl₃) [symbol] :42.4, 47.9, 52.6, 53.2, 62.8, 127.4, 128.4, 129.1, 132.1, 132.7, 133.6, 137.3, 147.6, 148.9, 170.0. LRMS: ES(+ve) m/z 360 (M+1). HRMS: CI(+ve) calculated for C₁₇H₁₉BrN₃O (M+H) 360.0708, found 360.0711.

### (4-(4-Bromobenzyl)piperazin-1-yl)(quinolin-3-yl)methanone (21)

General procedure A was applied to quinoline-3-carboxylic acid (3.45 g, 19.9 mmol) followed by column chromatography using CH₂Cl₂/CH₃OH (9:1) to give the title compound as a wax like solid (6.05 g, 74%). ¹H NMR (CDCl₃): δ 2.43 (br s, 2H, CH₂), 2.56 (br s, 2H, CH₂), 3.50 (s, 2H, CH₂), 3.52 (br s, 2H, CH₂), 3.75 (br s, 2H, CH₂), 7.20 (d, J=8.3 Hz, 2H, Ar), 7.45 (d, J=8.3 Hz, 2H, Ar), 7.61 (t, J=8.0 Hz, 1H, Ar), 7.78 (t, J=8.0 Hz, 1H, Ar), 7.86 (d, J=8.3 Hz, 1H, Ar), 8.13 (t, J=8.4 Hz, 1H, Ar), 8.24 (d, J=1.8 Hz, 1H, Ar), 8.94 (d, J=2.0 Hz, 1H, Ar). ¹³C NMR (CDCl₃): δ 43.5, 48.4, 54.2, 62.0, 127.4, 128.2, 129.4, 130.7, 135.2, 148.4, 130.6, 131.5, 127.5, 128.7, 136.5, 148.4, 167.8. LRMS: ES(+ve) m/z 434 (M+Na). HRMS: CI(+ve) calculated C₂₁H₂₀BrN₃ONa (M+Na) 434.0678, found 434.0678.

### General Procedures B and C for Stannylation

General procedure B: To a solution of the bromo compound (1.6 mmol) in anhydrous toluene (30 ml) was added tetrakis(triphenyl)phosphine palladium(0) (130 mg, 0.11 mmol) and hexamethylditin (800 mg, 2.5 mmol). The reaction mixture was heated to reflux for 24 h before another portion of hexamethylditin (510 mg, 1.6 mmol) was added. Reflux continued for another 24 h before the reaction mixture was cooled, filtered and the filtrate concentrated in vacuo. Purification of the crude residue by column chromatography gave the desired stannyl compound.

General procedure C: Sodium trimethyl stannane was prepared by adding hexamethylditin (750 mg, 2.3 mmol) in THF (3 ml) to a suspension of finely dispersed sodium (72 mg, 3.1 mmol) in THF (7 ml) at 0° C. After 3 h, the dark green reaction mixture was centrifuged for 20 sec at 2000 rpm then placed back in the ice bath. To a solution of the halogenated nicotinamide (0.8 mmol) in tetrahydrofuran (THF) (3 ml) at 0° C. under a nitrogen atmosphere was slowly added the supernatant (5 ml) of the centrifuged sodium trimethyl starmane solution. Stirring continued for 2 h and the reaction was then allowed to warm to room temperature. The reaction continued to stir overnight before it was diluted with ethyl acetate (20 ml) and washed with water (20 ml). The organic extract was dried (Na₂SO₄), concentrated in vacuo and then purified by column chromatography to give the desired stannyl compound.

### N-(2-(Diethylamino)ethyl)-5-(trimethylstannyl)nicotinamide (10)

General procedure B was applied to compound 9 (470 mg, 1.6 mmol) followed by column chromatography using CH₂Cl₂/CH₃OH (9:1) to give the title compound as an off-white wax like solid (370 mg, 61%). ¹H NMR (CDCl₃) [symbol] :0.37 (s, 9H, Sn(CH₃)₃), 1.11, (t, J=7.2 Hz, 3H, CH₃), 2.67, (q, J=7.2 Hz, 4H, 2×CH₂), 2.77 (t, J=5.6 Hz, 2H, CH₂), 3.56 (t, J=5.6 Hz, 2H, CH₂), 7.42 (br s, 1H, NH), 8.27 (t, J=1.2 Hz, 1H, Ar), 8.71 (d, J=1.6 Hz, 1H, Ar), 8.90 (d, J=2.4 Hz, 1H, Ar). ¹³C NMR (CDCl₃) [symbol] :−9.5, 11.6, 47.0, 51.5, 129.8, 142.4, 147.5, 157.8, 137.3, 166.0. LRMS: ES(+ve) m/z 386(M+1). HRMS: CI(+ve) calculated for C₁₅H₂₈N₃OSn (M+H) 386.1260, found 386.1255.

### N-(2-(Diethylamino)ethyl)-6-(trimethylstannyl)nicotinamide (13)

General procedure C was applied to compound 12 (200 mg, 0.78 mmol) to give a yellow oil that was further purified using a neutral alumina column (Brockman grade 1, 30×30 mm) (288 mg, 95%). ¹H NMR (d₄-CH₃OH) [symbol] :0.40 (s, 9H, Sn(CH₃)₃), 1.13 (t, J=7.2 Hz, 6H, 2×CH₃), 2.70 (q, J=7.2 Hz, 4H, 2×CH₂), 2.77 (t, J=7.2 Hz, 2H, CH₂), 3.55 (t, J=7.2 Hz, 2H, CH₂), 7.73 (d, J=8.0 Hz, 1H, Ar), 8.07 (dd, J=2.0, 8.0 Hz, 1H, Ar), 9.08 (d, J=2.0 Hz, 1H, Ar). ¹³C NMR (CDCl₃) [symbol] :−7.4, 13.6, 39.3, 48.5, 53.1, 130.1, 132.7, 133.5, 149.9, 166.8, 178.7. LRMS: ES(+ve) m/z 386 (M+1). HRMS: CI(+ve) calculated for C₁₅H₂₈N₃OSnNa (M+Na) 408.1079, found 408.1084.

### N-(4-(Dipropylamino)butyl)-5-(trimethylstannyl)nicotinamide (16)

General procedure B was applied to compound 15 (570 mg, 1.6 mmol) followed by column chromatography using CH₂Cl₂/CH₃OH (9:1) to give the title compound as a clear oil (285 mg, 41%). ¹H NMR (d₆-DMSO) [symbol] :0.34 (s, 9H, Sn(CH₃)₃), 0.81 (t, J=7.2 Hz, 6H, 2×CH₃), 1.37 (m, 4H, 2×CH₂), 1.40 (m, 2H, CH₂), 1.50 (m, 2H, CH₂), 2.28 (m, 4H, 2×CH₂), 2.35 (t, J=6.8 Hz, 2H, CH₂), 3.27 (dt, J=5.6, 6.8 Hz, 2H, CH₂), 8.21 (t, J=2.0 Hz, 1H, Ar), 8.59 (t, J=5.4 Hz, 1H, NH), 8.68 (d, J=2.0 Hz, 1H, Ar), 8.87 (d, J=2.0 Hz, 1H, Ar). ¹³C NMR (d₄-CH₃OH) δ:−9.9, 11.7, 19.5, 23.8, 28.0, 40.3, 54.2, 56.4, 131.9, 139.5, 143.9, 148.3, 157.7, 167.6. LRMS: ES(+ve) m/z 442 (M+1). HRMS: ES(+ve) calculated for C₁₉H₃₆N₃OSn (M+H) 442.1887, found 442.1884.

### (4-Benzylpiperazin-1-yl)(5-trimethylstannylpyridin-3-yl)methanone (19)

General procedure B was applied to compound 18 (570 mg, 1.6 mmol) followed by column chromatography using CH₂Cl₂/CH₃OH (9:1) to give the title compound as a clear oil (230 mg, 33%). ¹H NMR (CDCl₃) [symbol] :0.35 (s, 9H, Sn(CH₃)₃), 2.53 (br s, 2H, CH₂), 2.65 (br s, 2H, CH₂), 3.54 (br s, 2H, CH₂), 3.66 (s, 2H, CH₂), 3.88 (br s, CH₂), 7.27-7.37 (m, 5H, Ar), 7.83 (t, J=2.0 Hz, 1H, Ar), 8.54 (d, J=2.0 Hz, 1H, Ar), 8.65 (d, J=2.0 Hz, 1H, Ar). ¹³C NMR (CDCl₃) [symbol] :−8.21, 43.4, 48.5, 53.6, 54.1, 63.7, 129.1, 129.8, 130.7, 132.5, 137.6, 138.9, 143.5, 148.4, 157.7, 169.4. LRMS: ES(+ve) m/z 446 (M+1). HRMS: CI(+ve) calculated for C₂₀H₂₈N₃OSn (M+H) 446.1262, found 446.1272.

### (4-(4-(Trimethylstannyl)benzyl)piperazin-1-yl)(quinolin-3-yl)methanone (22)

Procedure C was applied to compound 21 (300 mg, 0.73 mmol) followed by column chromatography using CH₂Cl₂/CH₃OH (9:1) to give the title compound as an amber coloured oil (270 mg, 75%). ¹H NMR (CDCl₃): δ 0.30 (s, 9H, Sn(CH₃)₃), 2.43 (br s, 2H, CH₂), 2.57 (br s, 2H, CH₂), 3.57 (s, 2H, CH₂), 3.52 (br s, 2H, CH₂), 3.90 (br s, 2H, CH₂), 7.28 (d, J=7.5 Hz, 2H, Ar), 7.44 (d, J=7.4 Hz, 2H, Ar), 7.60 (t, J=7.2 Hz, 1H, Ar); 7.78 (t, J=7.5 Hz, 1H, Ar); 8.12 (d, J=8.4 Hz, 1H, Ar), 8.23 (s, 1H, Ar), 8.93 (d, J=1.7 Hz, 1H, Ar). ¹³C NMR (CDCl₃): δ −9.6, 50.8, 51.3, 52.3, 62.1, 63.4, 127.0, 127.5, 130.8, 135.2, 48.4, 128.3, 129.4, 128.7, 129.2, 133.7, 136.0, 148.3, 167.8. LRMS: BS(+ve) m/z 518 (M+Na). HRMS: CI(+ve) calculated for C₂₄H₂₉N₃OSnNa (M+Na) 518.1240, found 518.1235.

### General Procedure D for Preparation of Iodonicotinamides

To a solution of the stannane 10, 13, 16 or 19 (0.4 mmol) in chloroform (12 ml) was added iodine (0.4 mmol). The reaction was stirred at room temperature for 2 days before it was diluted with chloroform (40 ml) and then washed with a saturated solution of sodium bisulfite (40 ml). The organic extract was dried (Na₂SO₄) and concentrated in vacuo to give the desired iodo derivative.

### N-(2-(diethylamino)ethyl)-5-iodonicotinamide (11)

General procedure D was applied to compound 10 (150 mg, 0.39 mmol), followed by column chromatography with CH₂Cl₂/CH₃OH (8:2) to give the title compound as a wax like solid (80 mg, 59%). ¹H NMR (d₄-CH₃OH) [symbol] :1.11, (t, J=7.2 Hz, 3H, CH₃), 2.70 (q, J=7.2 Hz, 4H, 2×CH₂), 2.79, (t, J=5.7 Hz, 2H, CH₂), 3.60 (t, J=5.8 Hz, 2H, CH₂), 7.82 (br s, 1H, NH), 8.50 (t, J=1.58 Hz, 1H, Ar), 8.89 (d, J=2.0 Hz, 1H, Ar), 8.92 (d, J=1.7 Hz, 1H, Ar), ¹³C NMR (d₄-CH₃OH) [symbol] :12.8, 38.4, 48.0, 52.4, 94.5, 133.0, 144.7, 147.4, 159.3, 165.2. LRMS: ES(+ve) m/z 348 (M+1). Anal. calculated for C₁₂H₁₈IN₃O.1.9TFA C, H, N; 33.65, 3.56, 7.65; found 33.85, 3.63, 7.46.

### N-(2-(Diethylamino)ethyl)-6-iodonicotinamide (14)

General procedure D was applied to compound 13 (140 mg, 0.36 mmol), followed by HPLC purification (system A) eluting with H₂O/ACN/TFA, 80:20:0.1, v/v/v, to give the title compound as a clear oil (80 mg, 63%). ¹H NMR (d₄-CH₃OH) [symbol] :1.34 (t, J=7.31 Hz, 6H, 2×CH₃), 3.34 (m, 4H, 2×CH₂) 3.38 (t, J=6.1 Hz, 2H, CH₂), 3.75 (t, J=6.1 Hz, 2H, CH₂), 7.86 (dd, J=2.34, 8.15 Hz, 1H, Ar), 7.97 (d, J=8.15 Hz, 1H, Ar), 8.75 (d, J=2.34 Hz, 1H, Ar). ¹³C NMR (d₄-CH₃OH) [symbol] :8.9, 36.0, 48.8, 52.3, 122.2, 130.1, 136.2, 137.7, 150.3, 168.2. LRMS: ES(+ve) m/z 348 (M+1), HRMS: CI(+ve) calculated for C₁₂H₁₉IN₃O (M+H) 348.0573, found 348.0581.

### N-(4-(Dipropylamino)butyl)-5-iodonicotinamide (17)

General procedure D was applied to compound 16 (140 mg, 0.32 mmol) followed by column chromatography using CH₂Cl₂/CH₃OH (8:2) to give the title compound as a yellow oil (80 mg, 62%). ¹H NMR (CDCl₃) [symbol] :0.80 (t, J=7.4 Hz, 6H, 2×CH₃), 1.40 (m, 4H, 2×CH₂), 1.45 (m, 2H, CH₂), 2.38 (m, 4H, 2×CH₂), 2.45 (m, 2H, CH₂), 2.38 (t, J=6.5 Hz, 2H, CH₂), 8.45 (t, 1H, J=2.0 Hz, Ar), 8.83 (m, 2H, Ar). ¹³C NMR (CDCl₃) [symbol] :11.9, 20.1, 24.5, 28.1, 40.6, 54.5, 56.9, 93.6, 133.2, 144.7, 147.6, 158.6, 166.2. LRMS: ES(+ve) m/z 404 (M+1). HRMS: ES(+ve) calculated for C₁₆H₂₇IN₃O (M+H) 404.1193, found 404.1193.

### (4-Benzylpiperazin-1-yl)(5-iodopyridin-3-yl)methanone (20)

General procedure D was applied to compound 19 (175 mg, 0.4 mmol) followed by column chromatography using ethyl acetate/CH₃OH (9:1) to yield the title compound as a yellow oil (156 mg, 97%). ¹H NMR (CDCl₃) [symbol] :2.42 (br s, 2H, CH₂N), 2.54 (br s, 2H, CH₂N), 3.43 (br s, 2H, CH₂), 3.55 (s, 2H, CH₂), 3.78 (br s, 2H, CH₂), 7.26-7.35 (m, 5H, Ar), 8.07, (s, 1H, Ar), 8.57 (s, 1H, Ar), 8.86 (s, 1H, Ar). ¹³C NMR (CDCl₃) [symbol] :42.3, 47.8, 52.5, 53.1, 63.7, 93.2, 127.4, 128.4, 129.1, 133.2, 137.2, 143.1, 146.1, 156.7, 165.8. LRMS: ES(+ve) m/z 408 (M+1). HRMS: CI(+ve) calculated for C₁₇H₁₉IN₃O (M+H) 408.0573, found 408.561.

### (4-(4-Iodobenzyl)piperazin-1-yl)(quinolin-3-yl)methanone (23)

General Procedure D was applied to compound 22 (175 mg, 0.35 mmol) followed by column chromatography using CH₂Cl₂/CH₃OH (9:1) to give a wax like solid (120 mg, 74%). ¹H NMR (CDCl₃): δ 2.43 (br, 2H, CH₂), 2.54 (br, 2H, CH₂), 3.49 (br s, 2H, CH₂), 3.52 (br s, 2H, CH₂), 3.81 (br, 2H, CH₂), 7.07 (d, J=8.3 Hz, 2H, Ar), 7.64 (d, J=8.3 Hz, 2H, Ar), 7.61 (t, J=8.0 Hz, 1H, Ar), 7.78 (t, J=8.0 Hz, 1H, Ar), 7.85 (d, J=8.3 Hz, 1H, Ar), 8.13 (d, J=8.4 Hz, 1H, Ar), 8.23 (d, J=2.0 Hz, 1H, Ar), 8.94 (d, J=2.0 Hz, 1H, Ar). ¹³C NMR (CDCl₃): δ 43.8, 47.4, 54.5, 62.4, 128.3, 128.7, 129.5, 130.8, 135.2, 148.4, 137.5, 131.0, 127.5, 128.7, 137.3, 148.4, 167.8. LRMS: ES(+ve) m/z 458 (M+1). Anal. calculated for C₂₁H₂₀IN₃O C 55.15, H 4.41, N 9.19, found C 55.15, H 4.40, N 9.10.

Radiopharmaceutical Preparation with Na[I¹²³]

Peracetic acid (PAA) and chloramine-T (CAT) were used as oxidants for radiodination of the alkylamino nicotinamides 10, 13 and 16 and the benzylpiperazine 19 and 22. To a solution of 10, 13 or 16, (0.25 mg, 0.6 μmol) in acetic acid (200 μl) was added Na[¹²³I]I (0.5 GBq, 15 μl) and 10% PAA in acetic acid (100 μl). To a solution of the trimethylstannyl precursors 19 and 22 (0.25 mg, 0.6 μmol) in ethanol (200 μl) was added Na[¹²³I]I (0.5 GBq), CAT (4.5 mM, 100 μl) and HCl (1 M, 100 μl). After 5 min at room temperature, the radiolabelling reaction was quenched by adding Na₂S₂O₅ (260 mM, 100 μl) and NaHCO₃ (650 mM, 100 μl), followed by HPLC solvent (350 μl). The resulting solution was then purified by HPLC (Table 2). The radiolabelled compound was collected and dried in vacuo. The radioiodinated tracer was recovered with ethanol (100 μl) and then formulated in saline for biological evaluation.

TABLE 2
Radiolabelling data for the [¹²³I]MEL radiotracers

| | Purification | | | Retention | RCY |
|---|---|---|---|---|---|
| [¹²³I]Cmpd | Solvent ^{a} | Flow rate | | time (min) | % ^{d} |
| [¹²³I]11 | 20/80 | 2 | mL/min ^{b} | 16 | 50-70 |
| [¹²³I]14 | 20/80 | 1.5 | mL/min ^{b} | 16 | 30-40 |
| [¹²³I]17 | 30/70 | 1.5 | mL/min ^{b} | 15 | 20-26 |
| [¹²³I]20 | 45/55 | 3 | mL/min ^{c} | 10 | 24-30 |
| [¹²³I]23 | 50/50 | 3 | mL/min ^{b} | 17 | 75-85 |

^{a} Acetonitrile/Ammonium acetate solution 100 mM.
^{b} Phenomenex Bondclone C18 (10 μm, 7.8 × 300 mm).
^{c} Alltech Alpha bond C18 (10 μm, 10 × 250 mm).
^{d} Isolated yield (not decay corrected), specific activity > 2 GBq/nmol.

### Biological Data

Animal experiments were performed in compliance with the NHMRC Australian Code of Practice for the care and use of animals for scientific purposes. Female C57BL/6J black and BALB/c nude albino mice of 5 weeks age were obtained from the Animal Resources Centre, Western Australia. B16F0 murine melanoma cells were originally obtained from European Collection of Cell Cultures (UK) and A375 human amelanotic melanoma cells were originally obtained from American Type Culture Collection (USA). Before transplantation, B16F0 and A375 melanoma cells were maintained as a monolayer in RPMI culture medium supplemented with 10% foetal calf serum and antibiotics and passaged with trypsinisation. Early passages were frozen and stored in liquid nitrogen. Cells were passaged to P=10 and then discarded. Frozen aliquots were grown in a monolayer culture to between 80-95% confluence and for transplantation were trypsinised and washed with Ca²⁺ and Mg²⁺ free phosphate buffered saline (PBS). For inoculation, B16F0 melanoma cells were resuspended in Ca²⁺ and Mg²⁺ free PBS at 3 or 5×10⁶ viable cells per ml and 0.1 ml was subcutaneously injected at the left flank of 6-7 weeks old C57BL/6J mice. Eleven days later, tumours could be palpated in >98% of inoculated animals. A375 human melanoma cells were resuspended at 1×10⁷ viablecells per ml and 0.1 ml was injected subcutaneously at the left flank of 6 weeks old BALB/c nude mice and 25-26 days later tumours could be palpated with ˜60% of inoculated animals developing tumours.

Biodistribution Studies

Eleven days (B16F0 melanoma) and 25 days (A375 human melanoma) after tumour transplantation, the [¹⁸F]nicotinamides (0.5-1.5 MBq, 100 μl) and [¹²³I]nicotinamide (0.37-0.74 MBq, 100 μl) derivatives were injected intravenously via the tail vein into mice (15-18 g). Time points of 1, 3, 6, 24, 48, 72 h after injection were chosen for determining the distribution of each compound in various organs and tissues for the iodine-123 labelled compounds and between 15 min and 6 hours (e.g. 15 min, 30 min, 1 h, 2 h, 4 h or 6 h) were chosen for the ¹⁸F-labelled compounds. At defined times post injection, groups of mice (n=5) were weighed, sacrificed by CO₂ administration followed by cervical dislocation and dissected. Selected organs were weighed and their radioactivity measured with a γ-counter. The remaining activity in the carcass was also determined in order to obtain the total activity in the mouse at defined time points. The fraction of injected activity (% ID) in the organ was calculated by comparison with suitable dilutions of the injected dose. Then, the radioactivity concentration in the organ (% ID/g) was found by dividing the % ID for each organ by the weight of the organ. The results of the uptake of the various radiotracers are shown in the following tables.

TABLE 3
Biodistribution of [¹⁸F]MEL2 in B16 melanoma bearing mice

| B16 | 15 min | 30 min | 1 h | 2 h | 3 h | 6 h |
|---|---|---|---|---|---|---|
| Time | 0.25 | 0.5 | 1 | 2 | 3 | 6 |
| LIVER | 10.148 | 5.825 | 2.813 | 0.693 | 0.491 | 0.123 |
| SPLEEN | 8.585 | 7.225 | 2.393 | 1.848 | 0.890 | 0.470 |
| KIDNEY | 14.117 | 9.595 | 3.446 | 0.676 | 0.394 | 0.075 |
| MUSCLE | 3.796 | 2.240 | 1.248 | 0.276 | 0.201 | 0.032 |
| SKIN | 2.214 | 1.473 | 0.766 | 0.356 | 0.172 | 0.041 |
| BONE | 3.493 | 2.078 | 1.368 | 0.578 | 0.624 | 0.515 |
| LUNGS | 5.616 | 2.922 | 1.632 | 0.386 | 0.253 | 0.066 |
| HEART | 3.153 | 1.902 | 0.977 | 0.246 | 0.166 | 0.054 |
| BLOOD | 2.048 | 1.218 | 0.619 | 0.147 | 0.111 | 0.020 |
| STOMACH | 9.344 | 6.639 | 3.370 | 1.472 | 0.828 | 0.193 |
| GIT | 5.421 | 3.855 | 2.102 | 0.708 | 0.541 | 0.135 |
| BRAIN | 2.471 | 1.997 | 1.096 | 0.270 | 0.150 | 0.039 |
| THYROID | 5.515 | 2.537 | 3.327 | 0.609 | 0.488 | 0.168 |
| TUMOUR | 6.970 | 8.582 | 8.376 | 9.416 | 7.753 | 7.747 |
| EYES | 14.678 | 15.976 | 18.808 | 17.281 | 17.276 | 15.591 |

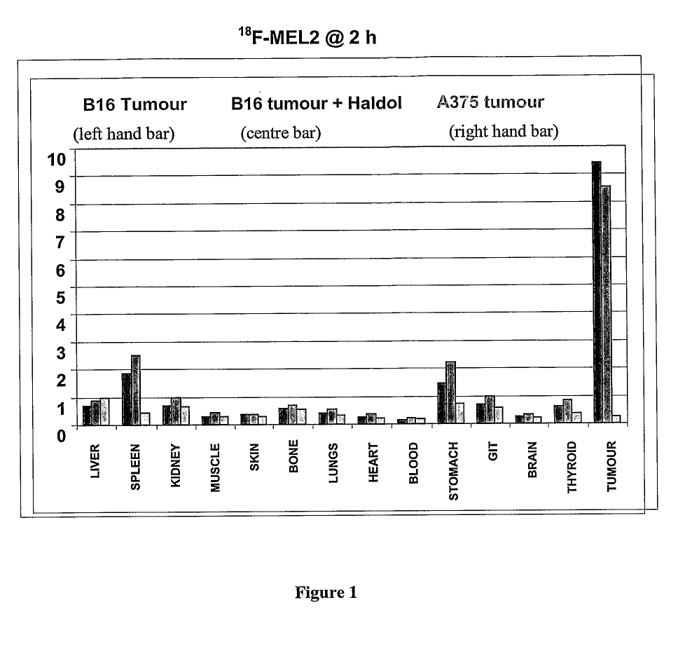

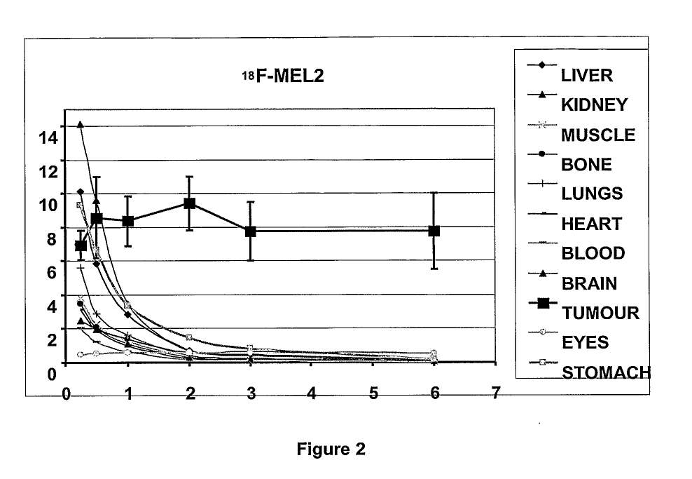

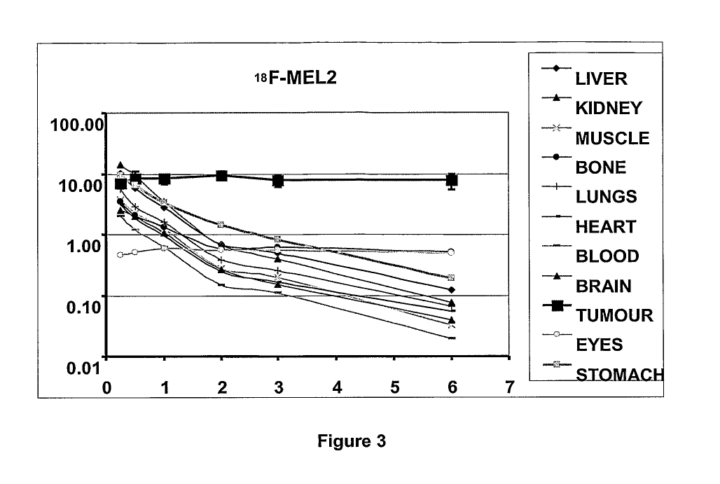

Table 3 shows the biodistribution of [¹⁸F]MEL2 in the main organs in B16 melanoma bearing mice over a six hour period. The key features of this distribution is the high uptake in melanin containing tissue (tumour and eyes) and rapid washout in all other tissue. The values are expressed as a percent of injected activity/gram of tissue. This is shown graphically in FIGS. 1-3.

TABLE 4
Biodistribution data of [¹⁸F]MEL6

| | TIME | | | |
|---|---|---|---|---|
| | 15 min | 1 h | 3 h | 6 h |
| LIVER | 12.2 | 4.9 | 1.9 | 1.0 |
| SPLEEN | 11.8 | 9.2 | 2.5 | 0.5 |
| KIDNEY | 16.4 | 4.0 | 0.8 | 0.2 |
| MUSCLE | 4.0 | 1.2 | 0.3 | 0.2 |
| SKIN | 2.7 | 1.0 | 0.2 | 0.9 |
| BONE | 4.8 | 3.4 | 4.6 | 5.6 |
| LUNGS | 5.0 | 1.5 | 0.3 | 0.2 |
| HEART | 3.7 | 1.2 | 0.3 | 0.2 |
| BLOOD | 2.8 | 0.9 | 0.2 | 0.1 |
| STOMACH | 14.7 | 6.8 | 1.9 | 0.7 |
| GIT | 7.8 | 7.1 | 7.1 | 3.6 |
| BRAIN | 6.2 | 1.6 | 0.4 | 0.1 |
| THYROID | 4.5 | 2.2 | 2.1 | 3.2 |
| TUMOUR | 10.6 | 14.6 | 17.3 | 4.8 |
| EYES | 36.7 | 38.0 | 36.3 | 27.4 |

Table 4 shows biodistribution of [¹⁸F]MEL6 in B16 melanoma bearing mice over a six hour period. The uptake values are expressed as a percent of injected activity/gram of tissue.

TABLE 5
Biodistribution data for [¹⁸F]MEL8

| | TIME | | | |
|---|---|---|---|---|
| | 15 min | 1 h | 3 h | 6 h |
| LIVER | 12.65 | 3.19 | 0.42 | 0.60 |
| SPLEEN | 10.27 | 3.19 | 2.32 | 1.31 |
| KIDNEY | 19.45 | 4.75 | 0.45 | 0.25 |
| MUSCLE | 4.16 | 1.27 | 0.28 | 0.17 |
| SKIN | 2.40 | 0.88 | 0.15 | 0.29 |
| BONE | 4.03 | 1.52 | 0.77 | 0.43 |
| LUNGS | 5.67 | 1.81 | 0.37 | 0.14 |
| HEART | 3.51 | 1.14 | 0.33 | 0.17 |
| BLOOD | 2.55 | 0.77 | 0.12 | 0.06 |
| STOMACH | 13.20 | 5.25 | 0.82 | 0.30 |
| GIT | 7.03 | 2.85 | 0.72 | 0.23 |
| BRAIN | 3.47 | 1.37 | 0.19 | 0.07 |
| THYROID | 4.81 | 2.22 | 1.48 | 1.02 |
| TUMOUR | 6.99 | 12.22 | 8.80 | 7.20 |
| EYES | 17.25 | 23.66 | 18.81 | 16.49 |

Table 5 shows biodistribution of [¹⁸F]MEL8 in B16 melanoma bearing mice over a six hour period. The uptake values are expressed as a percent of injected activity/gram of tissue.

TABLE 6
Biodistribution data for [¹⁸F]MEL4

| | 15 min | 1 h | 3 h | 6 h |
|---|---|---|---|---|
| LIVER | 30.69 | 36.76 | 32.81 | 26.41 |
| SPLEEN | 11.19 | 7.38 | 5.31 | 4.11 |
| KIDNEY | 15.88 | 4.64 | 2.89 | 1.80 |
| MUSCLE | 2.83 | 1.00 | 0.53 | 0.36 |
| SKIN | 2.53 | 1.61 | 1.09 | 0.89 |
| BONE | 3.89 | 2.03 | 1.41 | 1.03 |
| LUNGS | 12.06 | 4.49 | 3.08 | 2.23 |
| HEART | 2.49 | 0.98 | 0.71 | 0.52 |
| BLOOD | 0.68 | 0.32 | 0.21 | 0.14 |
| STOMACH | 8.52 | 3.31 | 2.36 | 1.79 |
| GIT | 8.23 | 5.78 | 4.18 | 3.08 |
| BRAIN | 0.84 | 0.75 | 0.57 | 0.37 |
| THYROID | 10.16 | 5.37 | 2.92 | 1.98 |
| TUMOUR | 4.01 | 4.79 | 5.31 | 5.29 |
| EYES | 11.27 | 12.65 | 14.28 | 15.75 |

Table 6 shows biodistribution of [¹⁸F]MEL4 in B16 melanoma bearing mice over a six hour period. The uptake values are expressed as a percent of injected activity/gram of tissue.

MEL11, 14, 17 and 20 are defined in the table below. Each compound was prepared both as the stable ¹²⁷I isotope, used as a standard to characterise, as well as the ¹²³I analogue which may be used in imaging or biodistribution studies.

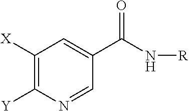

| Compound | X | Y | R |
|---|---|---|---|
| MEL11 | I | H | 2-(N,N-diethylamino)ethyl |
| MEL14 | H | I | 2-(N,N-diethylamino)ethyl |
| MEL17 | I | H | 4-(N,N-dipropylamino)butyl |
| MEL20 | I | H | 4-benzyl-piperazine |

TABLE 7
Biodistribution data of [¹²³I]MEL11, [¹²³I]MEL14, [¹²³I]MEL17 and [¹²³I]MEL20: Organ distribution in
percent of injected dose/organ (% ID/organ ± S.D.; n = 5) of [¹²³I]nicotinamides in C57BL/6J mice grafted with B16F0
melanoma tumour and in BALB/c nude mice grafted with A375 melanoma tumour.

| | Tumour | | | | | | | |
|---|---|---|---|---|---|---|---|---|
| Compound | cell line | Time (h) | Tumour | Thyroid | Spleen | Liver | Stomach | Intestine |
| [¹²³I]11 | B16 | 1 | 1.3 ± 0.3 | 0.37 ± 0.07 | 0.16 ± 0.16 | 1.8 ± 0.3 | 2.0 ± 0.2 | 15 ± 2 |
| | | 3 | 1.0 ± 0.4 | 1.1 ± 0.3 | 0.06 ± 0.05 | 0.82 ± 0.08 | 1.3 ± 0.3 | 5 ± 2 |
| | | 6 | 1.1 ± 0.3 | 1.8 ± 1.0 | 0.05 ± 0.04 | 0 53 ± 0.02 | 1.2 ± 0.2 | 2.8 ± 0.4 |
| | | 24 | 0.7 ± 0.1 | 1.0 ± 0.3 | 0.01 ± 0.03 | 0.12 ± 0.01 | 0.04 ± 0.01 | 0.10 ± 0.02 |
| | | 48 | 0.7 ± 0.4 | 1.4 ± 0.3 | 0.00 ± 0.00 | 0.06 ± 0.01 | 0.02 ± 0.01 | 0.06 ± 0.01 |
| | | 72 | 0.3 ± 0.1 | 1.0 ± 0.6 | 0.01 ± 0.01 | 0.04 ± 0.01 | 0.01 ± 0.01 | 0.04 ± 0.01 |
| | A375 | 1 | 0.40 ± 0.16 | 0.40 ± 0.07 | 0.16 ± 0.01 | 2.3 ± 0.2 | 1.6 ± 0.2 | 14 ± 2 |
| | | 6 | 0.13 ± 0.06 | 1.0 ± 0.5 | 0.03 ± 0.01 | 0.7 ± 0.2 | 0.4 ± 0.1 | 2 ± 1 |
| | | 24 | 0.001 | 1.4 ± 0.8 | 0.002 | 0.09 ± 0.02 | 0.01 ± 0.00 | 0.04 ± 0.01 |
| [¹²³I]14 | B16 | 1 | 1.8 ± 0.4 | 1.2 ± 0.7 | 0.31 ± 0.07 | 2.7 ± 0.7 | 9 ± 1 | 10 ± 1 |
| | | 6 | 1.4 ± 0.8 | 3.3 ± 0.8 | 0.17 ± 0.05 | 1.3 ± 0.4 | 7 ± 2 | 5 ± 1 |
| | | 24 | 0.11 ± 0.06 | 6.5 ± 2.8 | 0.01 ± 0.00 | 0.19 ± 0.04 | 0.4 ± 0.1 | 0.35 ± 0.03 |
| [¹²³I]17 | B16 | 1 | 0.64 ± 0.20 | 1.3 ± 0.4 | 0.54 ± 0.09 | 2.0 ± 0.1 | 6 ± 1 | 15 ± 1 |
| | | 6 | 1.0 ± 0.2 | 4.0 ± 0.9 | 0.13 ± 0.02 | 0.95 ± 0.15 | 6 ± 2 | 9 ± 1 |
| | | 24 | 0.62 ± 0.22 | 6.0 ± 1.6 | 0.02 ± 0.03 | 0.14 ± 0.03 | 0.17 ± 0.04 | 0.40 ± 0.07 |
| [¹²³I]20 | B16 | 1 | 2.6 ± 1.0 | 0.30 ± 0.08 | 0.05 ± 0.01 | 3.6 ± 0.9 | 1.7 ± 0.6 | 58 ± 2 |
| | | 6 | 1.7 ± 0.5 | 1.1 ± 0.4 | 0.04 ± 0.02 | 1.9 ± 0.4 | 4 ± 3 | 30 ± 3 |
| | | 24 | 0.36 ± 0.07 | 1.4 ± 0.6 | 0.00 ± 0.00 | 0.13 ± 0.04 | 0.07 ± 0.02 | 0.4 ± 0.1 |

TABLE 8
Biodistribution of [¹²³I]nicotinamides. Uptake in percent of injected dose per gram of tissue (% ID/g ± SD, n =
5) and calculated tumour standardized uptake values (SUVₜ).

| | Tu- | | | | | | | | | |
|---|---|---|---|---|---|---|---|---|---|---|
| | mour | | | | | | | | | |
| Com- | mod- | Time | | | | | | | | |
| pound | el | (h) | Tumour | Liver | Kidney | Lung | Heart | Brain | Blood | SUV³ |
| [¹²³I]11 | B16 | 1 | 7.8 ± 1.7 | 1.9 ± 0.3 | 1.8 ± 0.5 | 1.4 ± 0.2 | 0.8 ± 0.1 | 0.14 ± 0.02 | 1.21 ± 0.17 | 3.7 ± 1.0 |
| | | 3 | 6.0 ± 0.4 | 0.9 ± 0.1 | 0.8 ± 0.1 | 0.75 ± 0.07 | 0.4 ± 0.04 | 0.07 ± 0.01 | 0.92 ± 0.09 | 7.4 ± 1.6 |
| | | 6 | 5.9 ± 0.7 | 0.6 ± 0.1 | 0.5 ± 0.1 | 0.47 ± 0.11 | 0.26 ± 0.06 | 0.04 ± 0.01 | 0.58 ± 0.14 | 12 ± 1 |
| | | 24 | 3.2 ± 0.9 | 0.12 ± 0.02 | 0.03 ± 0.01 | 0.03 ± 0.02 | 0.04 ± 0.01 | 0.01 | 0.03 ± 0.01 | 37 ± 9 |
| | | 48 | 2.2 ± 0.4 | 0.08 ± 0.02 | 0.03 ± 0.01 | 0.04 ± 0.01 | 0.03 ± 0.01 | 0.01 | 0.03 v 0.01 | 22 ± 5 |
| | | 72 | 1.4 ± 0.3 | 0.04 ± 0.01 | 0.02 ± 0.02 | 0.04 ± 0.04 | 0.06 ± 0.02 | 0.01 | 0.02 ± 0.01 | 18 ± 12 |
| | A375 | 1 | 1.2 ± 0.2 | 2.2 ± 0.4 | 1.9 ± 0.3 | 1.56 ± 0.2 | 0.8 ± 0.1 | 0.14 ± 0.02 | 1.4 ± 0.2 | 0.7 ± 0.1 |
| | | 6 | 0.4 ± 0.1 | 0.7 ± 0.3 | 0.47 ± 0.17 | 0.4 ± 0.2 | 0.18 ± 0.07 | 0.03 ± 0.01 | 0.5 ± 0.2 | 0.9 ± 0.2 |
| | | 24 | 0.007 ± 0.002 | 0.08 ± 0.02 | 0.013 ± 0.002 | 0.015 ± 0.004 | 0.012 ± 0.003 | 0.002 ± 0.001 | 0.013 ± 0.002 | 0.3 ± 0.1 |
| [¹²³I]14 | B16 | 1 | 3.8 ± 0.2 | 3.1 ± 0.6 | 4.2 ± 0.6 | 4.8 ± 0.4 | 2.3 ± 0.2 | 0.36 ± 0.05 | 6.9 ± 0.6 | 1.2 ± 0.1 |
| | | 6 | 2.3 ± 0.5 | 1.4 ± 0.5 | 2.4 ± 0.7 | 2.6 ± 0.8 | 1.1 ± 0.3 | 0.14 ± 0.04 | 3.6 ± 1.0 | 1.0 ± 0.1 |
| | | 24 | 0.26 ± 0.14 | 0.20 ± 0.04 | 0.18 ± 0.05 | 0.2 ± 0.1 | 0.10 ± 0.05 | 0.02 | 0.25 ± 0.12 | 0.5 ± 0.2 |
| [¹²³I]17 | B16 | 1 | 5.6 ± 0.3 | 2.1 ± 0.2 | 3.5 ± 0.2 | 7.5 ± 0.2 | 1.9 ± 0.1 | 0.42 ± 0.02 | 3.1 ± 0.1 | 1.8 ± 0.1 |
| | | 6 | 5.0 ± 0.7 | 1.1 ± 0.1 | 1.7 ± 0.3 | 2.2 ± 0.2 | 0.8 ± 0.1 | 0.16 ± 0.03 | 2.4 ± 0.3 | 2.2 ± 0.4 |
| | | 24 | 2.3 ± 0.4 | 0.15 ± 0.03 | 0.12 ± 0.03 | 0.15 ± 0.3 | 0.09 ± 0.02 | 0.02 | 0.13 ± 0.03 | 5.1 ± 1.3 |
| [¹²³I]20 | B16 | 1 | 6.0 ± 1.6 | 3.6 ± 0.9 | 4.2 ± 0.6 | 0.90 ± 0.06 | 0.49 ± 0.03 | 0.20 ± 0.02 | 0.89 ± 0.07 | 1.5 ± 0.4 |
| | | 6 | 3.9 ± 0.9 | 2.2 ± 0.6 | 2.4 ± 0.4 | 0.46 ± 0.13 | 0.24 ± 0.08 | 0.05 ± 0.01 | 0.60 ± 0.17 | 1.7 ± 0.5 |
| | | 24 | 1.0 ± 0.2 | 0.12 ± 0.02 | 0.08 ± 0.01 | 0.03 ± 0.01 | 0.02 ± 0.01 | 0.001 | 0.04 ± 0.01 | 9 ± 2 |

Data are the means of % ID/g of tissue±SD, n=5, B16 melanoma tumour in C57BL/6J mice, A375 melanoma tumour in BALB/c nude mice, Standardised uptake values (SUVₜ) are calculated by dividing the tumour radioactivity concentration by the mean radioactive concentration remaining in the mouse at time t.

TABLE 9
HPLC Metabolite Analysis of [¹⁸F]MEL2.
Percentage Unchanged Tracer

| | Time (minutes) | | |
|---|---|---|---|
| Tissue | 15 | 60 | 120 |
| Plasma | | | |
| Metabolite 1 (0.8 min) | 10% | 14% | 25% |
| Metabolite 2 (3.0 min) | | | |
| Metabolite 3 (6 min) | 5% | 7% | 12% |
| Unchanged | 85% | 79% | 63% |
| Tumour | | | |
| Polar Metabolite 1 | | | 2% |
| Polar Metabolite 3 | | | |
| Unchanged | 100% | 100% | 98% |
| Eye | | | |
| Polar Metabolite 1 (0.8 min) | | 4% | 1% |
| Polar Metabolite 2 (3.0 min) | | | |
| Unchanged | 100% | 96% | 99% |
| Urine | | | |
| Polar Metabolite 1 (0.8 min) | | 5% | 8% |
| Polar Metabolite 2 (3.0 min) | | | |
| Polar Metabolite 3 (6 min) | | 5% | 5% |
| Unchanged | | 90% | 87% |

Table 9 Shows the HPLC analysis of [¹⁸F]MEL2 in Plasma, Tumour, Eye and Urine. Analysis of the unchanged tracer [¹⁸9MEL2 at various time points indicates that the uptake of activity in the tumour and eye is unchanged [¹⁸F]MEL2 and not metabolites of [¹⁸F]MEL2. In urine C⁸F]MEL2 is excreted predominately as the unchanged radiotracer at all time points studied.

TABLE 10
Biodistribution of [¹⁸F]MEL2 in A375 melanotic tumours

| | 30 min | 1 h | 2 h | 3 h |
|---|---|---|---|---|
| LIVER | 6.820 | 2.670 | 0.985 | 0.323 |
| SPLEEN | 4.093 | 1.351 | 0.434 | 0.103 |
| KIDNEY | 7.103 | 2.032 | 0.650 | 0.197 |
| MUSCLE | 2.125 | 0.865 | 0.280 | 0.108 |
| SKIN | 2.521 | 1.265 | 0.290 | 0.169 |
| BONE | 1.912 | 0.863 | 0.548 | 0.443 |
| LUNGS | 2.836 | 0.939 | 0.324 | 0.099 |
| HEART | 1.898 | 0.604 | 0.205 | 0.045 |
| BLOOD | 1.323 | 0.480 | 0.171 | 0.054 |
| URINE | 609.148 | 185.427 | 97.912 | 37.842 |
| BLADDER | 8.937 | 3.717 | 4.835 | 0.360 |
| STOMACH | 3.795 | 1.899 | 0.748 | 0.302 |
| GIT | 3.432 | 1.564 | 0.580 | 0.227 |
| TAIL | | | | |
| BRAIN | 1.831 | 0.822 | 0.224 | 0.073 |
| THYROID | 2.745 | 1.162 | 0.356 | 0.128 |
| TUMOUR | 2.633 | 0.920 | 0.254 | 0.096 |
| EYES | 1.704 | 0.626 | 0.238 | 0.110 |

Table 10 shows the biodistribution of [¹⁸F]MEL2 in the main organs in A375 amelanotic melanoma tumour bearing mice over a three hour period. The key features of this distribution is the low uptake in tissue (tumour and eyes) of [¹⁸F]MEL2. When comparing to table 3 (biodistribution of [¹⁸F]MEL2 in B 16 melanoma bearing mice—B16 is a melanin containing tumour), there is a large uptake difference (between B16 and A375 animal models). This supports the hypothesis that [¹⁸F]MEL2 is involved in specific interaction with melanin which is inherent with the pigmented eye structure of C57BL/6J black mice and the B16 tumour.

1. A compound comprising a pyridine carboxamide structure wherein an aromatic ring in the structure is substituted with a radiohalogen atom, wherein the radiohalogen atom is ¹⁸F and wherein the substitution on the amide nitrogen atom is:
a hydrogen atom and a tertiary aminoalkyl group; or
such that the amide nitrogen is a member of a saturated ring structure having a second nitrogen atom in the ring;
such that the compound binds to melanin, or a pharmaceutically acceptable salt of said compound.

2. The compound or salt of claim 1 wherein the aromatic ring that is substituted with the radiohalogen atom is the pyridine ring of the pyridine carboxamide structure.

3. The compound or salt of claim 1 wherein the second nitrogen atom in the saturated ring structure is substituted with an arylalkyl group.

4. The compound or salt of claim 3 wherein the aromatic ring that is substituted with the radiohalogen atom is the aryl group of the arylalkyl group.

5. The compound or salt of claim 1 wherein the pyridine carboxamide structure is a pyridine-3-carboxamide structure.

6. The compound or salt of any one of claims 1 to 5 wherein the pyridine ring of the pyridine carboxamide structure is fused with a benzene ring to form a quinoline ring system.

7. The compound or salt of claim 1 wherein the compound has the structure 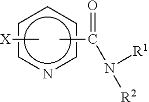
wherein X is ¹⁸F and R¹ is hydrogen and R² is a tertiary alkyl group such that the compound is capable of binding to melanin.

8. The compound or salt of claim 1 wherein the compound has structure 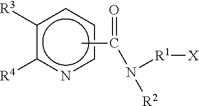
wherein:
X is ¹⁸F,
R¹ and R² together with the amide nitrogen form a piperazine ring, said piperazine ring being substituted with a benzyl group on the non-amide nitrogen such that the compound is capable of binding to melanin, wherein the radiohalogen atom is attached to the benzyl group; and R³ and R⁴ together form a ring fused with the pyridine ring.

9. A process for making a compound, said compound comprising a pyridine carboxamide structure wherein an aromatic ring in the structure is substituted with a radiohalogen atom, wherein the radiohalogen atom is ¹⁸F and wherein the substitution on the amide nitrogen atom is a hydrogen atom and a tertiary aminoalkyl group such that the compound binds to melanin, or a pharmaceutically acceptable salt of said compound,
the process comprising the step of treating a precursor comprising a leaving group so as to replace said leaving group with the radiohalogen atom ¹⁸F, said precursor comprising a pyridine carboxamide structure wherein an aromatic ring in the structure is substituted with said leaving group and wherein the substitution on the amide nitrogen atom is a hydrogen atom and a tertiary aminoalkyl group, such that the compound binds to melanin.

10. The process of claim 9 wherein the leaving group is a non-radioactive halogen atom.

11. The process of claim 10 wherein the non-radioactive halogen is chlorine or bromine.

12. The process of claim 9 wherein the step of treating the precursor comprises treating the precursor with a complex of M⁺[¹⁸F⁻] in the presence of a metal complexing agent, wherein M⁺ is a metal ion which is either sufficiently large to allow substitution of the leaving group with ¹⁸F⁻ or is complexed with a complexing agent so as to allow substitution of the leaving group with ¹⁸F⁻.

13. The process of claim 12 wherein the complex of M⁺[¹⁸F⁻] is K[¹⁸F⁻] K_{2.2.2.}K₂CO₃ complex or a tetrabutylamonium [¹⁸F] fluoride complex.

14. The process of claim 9 wherein the step of treating the precursor comprises:
substituting the leaving group by an organometallic group and
substituting the organometallic group by the radiohalogen halogen atom, wherein the radiohalogen atom is ¹⁸F.

15. The process of claim 14, wherein the source of the radiohalogen atom ¹⁸F₂ or [¹⁸F]acetyl hypofluorite.

16. The process of claim 14, wherein the organometallic group is an alkyl tin group.

17. The process of claim 9 wherein the final chemical step of the process comprises introducing the radiohalogen atom ¹⁸F into the compound.

18. The process of claim 17 wherein said final chemical step takes less than about 1 hour.

19. The process of claim 9 wherein the radiochemical yield of the total synthesis is higher than for the corresponding benzamides.

20. The process of claim 10 wherein the radiochemical yield of the total synthesis is greater than about 30%.

21. The process of claim 20, wherein the radiochemical yield of the total synthesis is greater than about 50%.

22. A process for making a compound, said compound comprising a pyridine carboxamide structure wherein an aromatic ring in the structure is substituted with a radiohalogen atom, wherein the radiohalogen atom is ¹⁸F and wherein the substitution on the amide nitrogen atom is a hydrogen atom and a tertiary aminoalkyl group such that the compound binds to melanin, or a pharmaceutically acceptable salt of said compound,
the process including the step of treating a solution of a precursor comprising a chloro leaving group in dimethylformamide with K[¹⁸F]-K_{2.2.2.}K₂CO₃ and heating the mixture at 150° C. for 10 minutes;
said precursor comprising a pyridine carboxamide structure wherein an aromatic ring in the structure is substituted with said leaving group and wherein the substitution on the amide nitrogen atom is a hydrogen atom and a tertiary aminoalkyl group, such that the compound binds to melanin.

23. The process of claim 22 wherein the radiochemical yield of the process is greater than about 30%.

24. A compound being made by the process of claim 9.

25. A compound according to claim 1 when used in imaging melanoma.

26. A method for imaging a melanoma in a patient, said method comprising:
administering to said patient a compound or salt according to claim 1;
allowing sufficient time for a PET-imageable quantity of the compound or salt to accumulate in said melanoma; and
imaging the melanoma using PET.

27. A composition for use in imaging melanoma, said composition comprising a compound or salt according to claim 1, together with one or more pharmaceutically acceptable carriers and/or adjuvants.

28. Use of a compound or salt according to claim 1 for the manufacture of a medicament for the imaging of melanoma.
